(19) United States
(12) Patent Application Publication
(10) Pub. No.: US 2002/0061576 A1
(43) Pub. Date: May 23, 2002

(54) TISSUE PLASMINOGEN ACTIVATOR-LIKE PROTEASE

(76) Inventors: PAUL A. MOORE, MCLEAN, VA (US); STEVEN M. RUBEN, OLNEY, MD (US); REINHARD EBNER, GAITHERSBURG, MD (US)

Correspondence Address: HUMAN GENOME SCIENCES INC, 9410 KEY WEST AVENUE, ROCKVILLE, MD, 20850

(21) Appl. No.: 09/084,491

(22) Filed: May 27, 1998

Related U.S. Application Data
(60) Provisional application No. 60/048,000, filed on May 28, 1997.

(51) Int. Cl. C07H 21/04

(52) U.S. Cl. 435/217; 435/212; 435/252.3; 435/252.33; 435/320.1; 536/23.2; 536/23.5

(57) ABSTRACT

The present invention relates to a novel t-PALP protein which is a member of the serine protease family. In particular, isolated nucleic acid molecules are provided encoding the human t-PALP protein. t-PALP polypeptides are also provided as are vectors, host cells and recombinant methods for producing the same. The invention further relates to screening methods for identifying agonists and antagonists of t-PALP activity. Also provided are diagnostic methods for detecting circulatory system-related disorders and therapeutic methods for treating circulatory system-related disorders.

### PRIORITY

This application claims benefit of 35 U.S.C. section 119(e) based on copending U.S. Provisional Application Ser. No. 60/048,000, filed May 28, 1997, herein incorporated by reference in its entirety.

### FIELD OF THE INVENTION

The present invention relates to a novel human gene encoding a polypeptide which is a homolog of tissue-type plasminogen activator (t-PA). More specifically, isolated nucleic acid molecules are provided encoding a human polypeptide named tissue-plasminogen activator-like protease, hereinafter referred to as “t-PALP”. t-PALP polypeptides are also provided, as are vectors, host cells and recombinant methods for producing the same. Also provided are diagnostic methods for detecting disorders related to the circulatory system and therapeutic methods for treating such disorders. The invention further relates to screening methods for identifying agonists and antagonists of t-PALP activity.

### BACKGROUND OF THE INVENTION

The plasmin coagulation system is activated in response to vascular injury. Within a few minutes of the injury, prothrombin is activated through the coagulation cascade to give rise to thrombin. Thrombin then converts fibrinogen to insoluble fibrin, which then interdigitates with and strengthens the primary platelet. Abnormal blood clotting can lead to many vascular diseases, such as stroke, deep-vein thrombosis, peripheral arterial occlusion, pulmonary embolism, and myocardiothrombosis, each of which constitutes a major health risk. Such diseases are primarily caused by partial or total occlusion of a blood vessel by a blood clot. Such clots consist essentially of a mass of fibrin and platelets. The prevention of clot formation and the dissolution of existing clots are two major therapeutic avenues frequently used for the treatment of disease states related to blood clots. Prevention of clot formation is primarily achieved through the inhibition of thrombin activity, whereas the dissolution of existing clots is frequently achieved by the activation of plasminogen which dissolves the existing blood clot (thereby affecting the fibrinolysis pathway).

The fibrinolytic system is activated by the deposition of fibrin. The conversion of fibrinogen to fibrin results in the exposure of many lysine residues on the surface of the molecule. A factor released from endothelial cells, termed tissue-type plasminogen activator (t-PA), activates plasminogen. Only upon activation can plasminogen bind to exposed lysine residues on the surface of fibrin, resulting in the degradation of fibrin, and, ultimately, the degradation of the blood clot itself.

In man and other animals, t-PA plays an essential role in the dissolution of fibrin clots (see, e.g., Verstraete and Collen, (1986) Blood 67:1425). t-PA is composed of several domains which share sequence homology with other proteins. These are the fibronectin finger-like domain, the epidermal growth factor-like domain, the kringle domain (of which t-PA has two), and the protease domain (Pennica, D., et al., (1983) Nature 301:214-221; Banyai, L., et al., (1983) FEBS Lett. 163:37-41). Only the function of the protease domain (residues 276-527) has been unambiguously defined. This finding was first based on the observed sequence homology with other known serine proteases. More recently, limited reduction of the two-chain form of t-PA has allowed the direct isolation and functional characterization of the protease region (Rijken and Groeneveld, (1986) J. Biol. Chem., 261:3098).

There is a clear need, therefore, for identification and characterization for such enzymes that influence the fibrinolytic system, both normally and in disease states. In particular, there is a need to isolate and characterize additional human tissue plasminogen activator and related protease-like molecules which possess such functions as the activation of plasminogen and may be employed, therefore, for preventing, ameliorating or correcting dysfunctions or disease states or, alternatively, augmenting the positive, natural actions of such enzymes.

### SUMMARY OF THE INVENTION

The present invention provides isolated nucleic acid molecules comprising a polynucleotide encoding at least a portion of the t-PALP polypeptide having the complete amino acid sequence shown in SEQ ID NO:2 or the complete amino acid sequence encoded by the cDNA clone deposited as plasmid DNA as ATCC Deposit Number 209023 on May 8, 1997. The nucleotide sequence determined by sequencing the deposited t-PALP clone, which is shown in FIG. 1 (SEQ ID NO:1), contains an open reading frame encoding a complete polypeptide of 263 amino acid residues, including an initiation codon encoding an N-terminal methionine at nucleotide positions 124-126, and a predicted molecular weight of about 28.2 kDa. Nucleic acid molecules of the invention include those encoding the complete amino acid sequence excepting the N-terminal methionine shown in SEQ ID NO:2, or the complete amino acid sequence excepting the N-terminal methionine encoded by the cDNA clone in ATCC Deposit Number 209023, which molecules also can encode additional amino acids fused to the N-terminus of the t-PALP amino acid sequence.

The t-PALP protein of the present invention shares sequence homology with the translation product of the human mRNA for t-PA (FIG. 2) (SEQ ID NO:3), including the following conserved domains: (a) the predicted kringle domain of about 60 amino acids and (b) the predicted protease domain of about 179 amino acids. t-PA is thought to be important in the regulation of blood clotting and disorders related thereto. The homology between t-PA and t-PALP indicates that t-PALP may also be involved in the regulation of normal and abnormal clotting in such conditions including many vascular diseases, such as stroke, deep-vein thrombosis, peripheral arterial occlusion, pulmonary embolism, and myocardiothrombosis.

The encoded polypeptide has a predicted leader sequence of about 21 amino acids underlined in FIG. 1. The amino acid sequence of the predicted mature t-PALP protein is also shown in FIG. 1, as amino acid residues 22-263 and as residues 1-242 in SEQ ID NO:2.

Thus, one aspect of the invention provides an isolated nucleic acid molecule comprising a polynucleotide having a nucleotide sequence selected from the group consisting of: (a) a nucleotide sequence encoding a full-length t-PALP polypeptide having the complete amino acid sequence in SEQ ID NO:2 excepting the N-terminal methionine (i.e., positions −20 to 242 of SEQ ID NO:2) or the complete amino acid sequence excepting the N-terminal methionine encoded by the cDNA clone contained in the ATCC Deposit No. 209023; (b) a nucleotide sequence encoding a mature t-PALP polypeptide having the amino acid sequence in SEQ ID NO:2 from residue 1 to 242 or as encoded by the cDNA clone contained in the ATCC Deposit No. 209023; (c) a nucleotide sequence encoding the predicted kringle domain of the t-PALP polypeptide having the amino acid sequence at positions 4 to 63 in SEQ ID NO:2 or as encoded by the cDNA clone contained in the ATCC Deposit No. 209023; (d) a nucleotide sequence encoding a polypeptide comprising the predicted protease domain of the t-PALP polypeptide having the amino acid sequence at positions 64 to 242 in SEQ ID NO:2 or as encoded by the cDNA clone contained in the ATCC Deposit No. 209023; and (e) a nucleotide sequence complementary to any of the nucleotide sequences in (a), (b), (c) or (d) above.

Further embodiments of the invention include isolated nucleic acid molecules that comprise a polynucleotide having a nucleotide sequence at least 90% identical (or 10% different), and more preferably at least 95%, 96%, 97%, 98% or 99% identical (or 5%, 4%, 3%, 2% or 1% different from), to any of the nucleotide sequences in (a), (b), (c), (d) or (e) above, or a polynucleotide which hybridizes under stringent hybridization conditions to a polynucleotide in (a), (b), (c), (d) or (e) above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a t-PALP polypeptide having an amino acid sequence in (a), (b), (c) or (d) above.

The present invention also relates to recombinant vectors, which include the isolated nucleic acid molecules of the present invention, and to host cells containing the recombinant vectors, as well as to methods of making such vectors and host cells and for using them for production of t-PALP polypeptides or peptides by recombinant techniques.

The invention further provides an isolated t-PALP polypeptide comprising an amino acid sequence selected from the group consisting of: (a) the amino acid sequence of the full-length t-PALP polypeptide having the complete amino acid sequence shown in SEQ ID NO:2 excepting the N-terminal methionine (i.e., positions −20 to 242 of SEQ ID NO:2) or the complete amino acid sequence excepting the N-terminal methionine encoded by the cDNA clone contained in the ATCC Deposit No. 209023; (b) the amino acid sequence comprising the mature form of the t-PALP polypeptide having the amino acid sequence at positions 1 to 242 in SEQ ID NO:2 or as encoded by the cDNA clone contained in the ATCC Deposit No. 209023; (c) the amino acid sequence comprising the predicted kringle domain of the t-PALP polypeptide having the amino acid sequence at positions 4 to 63 in SEQ ID NO:2 or as encoded by the cDNA clone contained in the ATCC Deposit No. 209023; and (d) the amino acid sequence comprising the predicted protease domain of the t-PALP polypeptide having the amino acid sequence at positions 64 to 242 in SEQ ID NO:2 or as encoded by the cDNA clone contained in the ATCC Deposit No. 209023. The polypeptides of the present invention also include polypeptides having an amino acid sequence at least 80% identical (that is, 20% different), more preferably at least 90% identical (10% different), and still more preferably 95%, 96%, 97%, 98% or 99% identical to (which also may be expressed as 5%, 4%, 3%, 2% or 1% different from) those described in (a), (b), (c) or (d) above, as well as polypeptides having an amino acid sequence with at least 90% similarity, and more preferably at least 95% similarity, to those above.

An additional embodiment of this aspect of the invention relates to a peptide or polypeptide which comprises the amino acid sequence of an epitope-bearing portion of a t-PALP polypeptide having an amino acid sequence described in (a), (b) or (c) above. Peptides or polypeptides having the amino acid sequence of an epitope-bearing portion of a t-PALP polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention.

In another embodiment, the invention provides an isolated antibody that binds specifically to a t-PALP polypeptide having an amino acid sequence described in (a), (b), (c) or (d) above. The invention further provides methods for isolating antibodies that bind specifically to a t-PALP polypeptide having an amino acid sequence as described herein. Such antibodies are useful diagnostically or therapeutically as described below.

The invention also provides for pharmaceutical compositions comprising t-PALP polypeptides, particularly human t-PALP polypeptides, which may be employed, for instance, to treat many vascular diseases, such as stroke, deep-vein thrombosis, peripheral arterial occlusion, pulmonary embolism, and myocardiothrombosis. Further uses of t-PALP may include induction of growth of hepatocytes and regeneration of liver tissue. Methods of treating individuals in need of t-PALP polypeptides are also provided.

The invention further provides compositions comprising a t-PALP polynucleotide or an t-PALP polypeptide for administration to cells in vitro, to cells ex vivo and to cells in vivo, or to a multicellular organism. In certain particularly preferred embodiments of this aspect of the invention, the compositions comprise a t-PALP polynucleotide for expression of a t-PALP polypeptide in a host organism for treatment of disease. Particularly preferred in this regard is expression in a human patient for treatment of a dysfunction associated with aberrant endogenous activity of a t-PALP.

The present invention also provides a screening method for identifying compounds capable of enhancing or inhibiting a biological activity of the t-PALP polypeptide, which involves contacting an enzyme which is activated by the t-PALP polypeptide with the candidate compound in the presence of a t-PALP polypeptide, assaying proteolytic activity of the plasminogen-like molecule in the presence of the candidate compound and of t-PALP polypeptide, and comparing the plasminogen-like molecule activity to a standard level of activity, the standard being assayed when contact is made between the plasminogen-like molecule and in the presence of the t-PALP polypeptide and the absence of the candidate compound In this assay, an increase in plasminogen-like molecule activity over the standard indicates that the candidate compound is an agonist of t-PALP activity and a decrease in plasminogen-like molecule activity compared to the standard indicates that the compound is an antagonist of t-PALP activity.

In another aspect, a screening assay for agonists and antagonists is provided which involves determining the effect a candidate compound has on t-PALP binding to a plasminogen-like molecule. In particular, the method involves contacting the plasminogen-like molecule with a t-PALP polypeptide and a candidate compound and determining whether t-PALP polypeptide binding to the plasminogen-like molecule is increased or decreased due to the presence of the candidate compound. In this assay, an increase in binding of t-PALP over the standard binding indicates that the candidate compound is an agonist of t-PALP binding activity and a decrease in t-PALP binding compared to the standard indicates that the compound is an antagonist of t-PALP binding activity.

It has been discovered that t-PALP is expressed not only in activated monocytes, but in a number of other cells and tissues including cerebellum, smooth muscle, resting and PHA-treated T-cells, GM-CSF-treated macrophages, frontal cortex of the brain, breast lymph node, chronic lymphocytic leukemic spleen, and several others. Therefore, nucleic acids of the invention are useful as hybridization probes for differential identification of the tissue(s) or cell type(s) present in a biological sample. Similarly, polypeptides and antibodies directed to those polypeptides are useful to provide immunological probes for differential identification of the tissue(s) or cell type(s). In addition, for a number of disorders of the above tissues or cells, particularly of the circulatory system, significantly higher or lower levels of t-PALP gene expression may be detected in certain tissues (e.g., cancerous and wounded tissues) or bodily fluids (e.g., serum, plasma, urine, synovial fluid or spinal fluid) taken from an individual having such a disorder. relative to a “standard” t-PALP gene expression level, i.e., the t-PALP expression level in healthy tissue from an individual not having the circulatory system disorder. Thus, the invention provides a diagnostic method useful during diagnosis of such a disorder, which involves: (a) assaying t-PALP gene expression level in cells or body fluid of an individual; (b) comparing the t-PALP gene expression level with a standard t-PALP gene expression level, whereby an increase or decrease in the assayed t-PALP gene expression level compared to the standard expression level is indicative of disorder in the circulatory system.

A further aspect of the invention is related to the relative clot-specificities which t-PALP and t-PA may possess. For example, t-PALP may have a higher or lower affinity for exerting its proteolytic activity with respect to a blood clot which localized itself to the lungs than does t-PA. In addition, t-PALP may have a higher or lower affinity for a specific constituent of a given blood clot than does t-PA. Thus. the t-PALP molecule may prove useful as an agent which, directly or indirectly, results in the dissolution of a blood clot with a higher or lower activity than other agents.

An additional aspect of the invention is related to a method for treating an individual in need of an increased level of t-PALP activity in the body comprising administering to such an individual a composition comprising a therapeutically effective amount of an isolated t-PALP polypeptide of the invention or an agonist thereof.

A still further aspect of the invention is related to a method for treating an individual in need of a decreased level of t-PALP activity in the body comprising, administering to such an individual a composition comprising a therapeutically effective amount of an t-PALP antagonist. Preferred antagonists for use in the present invention are t-PALP-specific antibodies.

### BRIEF DESCRIPTION OF THE FIGURES

FIG. 1 shows the nucleotide sequence (SEQ ID NO:1) and deduced amino acid sequence (SEQ ID NO:2) of t-PALP.

The predicted leader sequence of about 21 amino acids is underlined. Note that the methionine residue at the beginning of the leader sequence in FIG. 1 is shown in position number (positive) 1, whereas the leader positions in the corresponding sequence of SEQ ID NO:2 are designated with negative position numbers. Thus, the leader sequence positions 1 to 21 in FIG. 1 correspond to positions −21 to −1 in SEQ ID NO:2.

FIG. 2 shows the regions of identity between the amino acid sequences of the t-PALP protein and translation product of the human mRNA for t-PA (SEQ ID NO:3), determined by the computer program Bestfit (Wisconsin Sequence Analysis Package, Version 8 for Unix, Genetics Computer Group, University Research Park, 575 Science Drive, Madison, Wis. 53711) using the default parameters.

FIG. 3 shows an analysis of the t-PALP amino acid sequence. Alpha, beta, turn and coil regions; hydrophilicity and hydrophobicity; amphipathic regions; flexible regions; antigenic index and surface probability are shown. In the “Antigenic Index—Jameson-Wolf” graph, the positive peaks indicate locations of the highly antigenic regions of the t-PALP protein, i.e., regions from which epitope-bearing peptides of the invention can be obtained.

### DETAILED DESCRIPTION

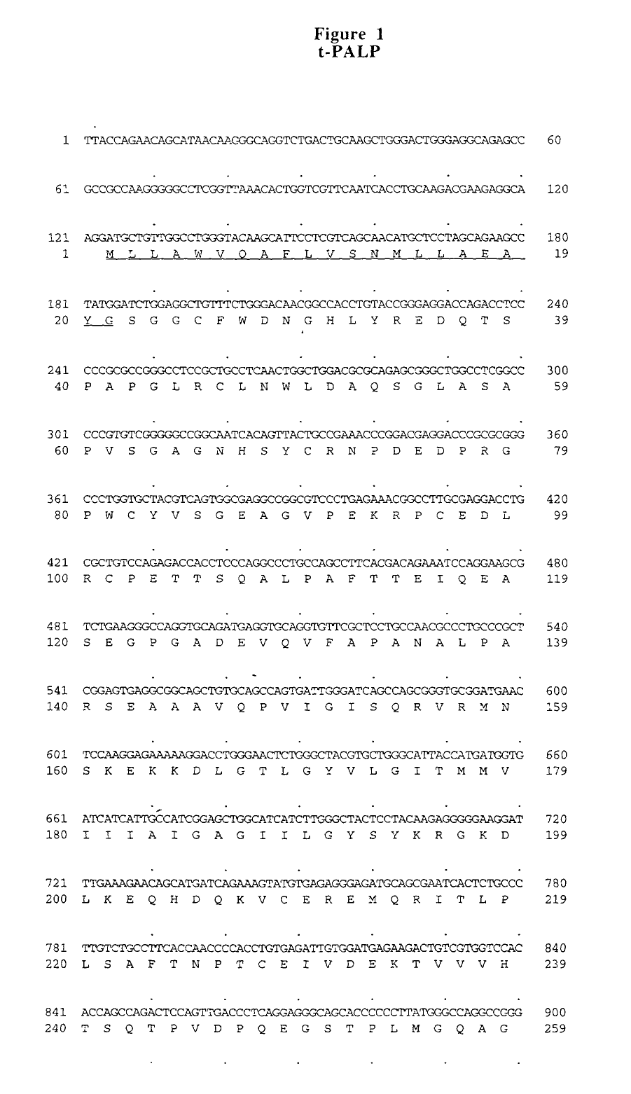

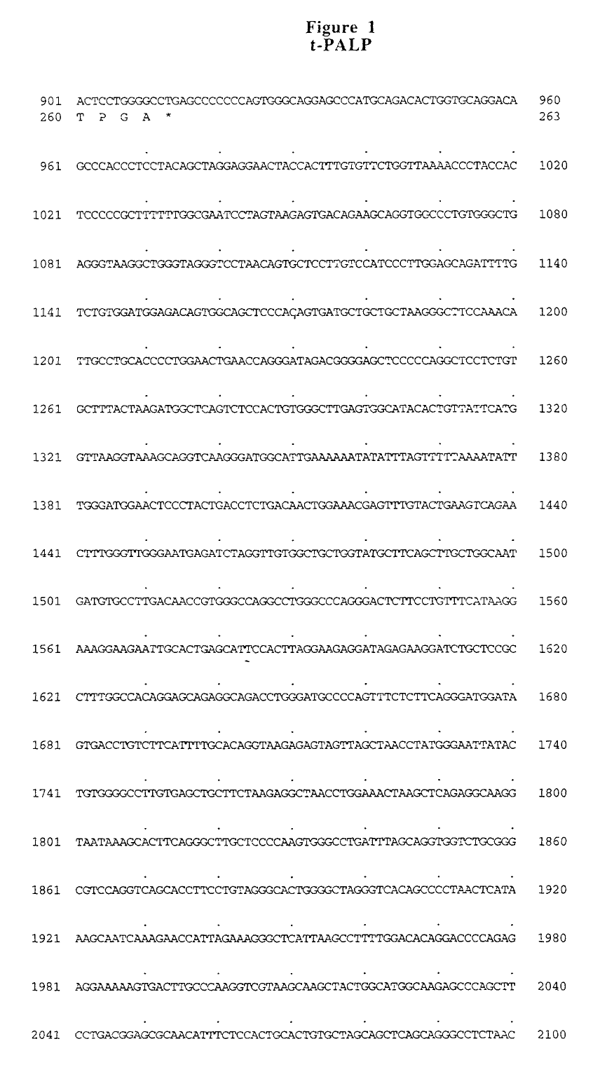

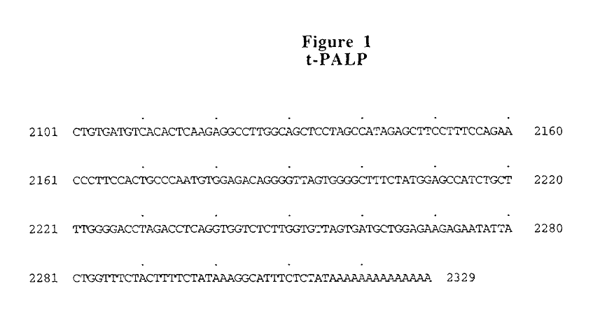

The present invention provides isolated nucleic acid molecules comprising a polynucleotide encoding a t-PALP polypeptide having the amino acid sequence shown in SEQ ID NO:2, which was determined by sequencing a cloned cDNA. The nucleotide sequence shown in FIG. 1 (SEQ ID NO:1) was obtained by sequencing the HMSIB42 clone, which was deposited on May 8, 1997 at the American Type Culture Collection, 12301 Park Lawn Drive, Rockville, Md. 20852, and given accession number ATCC 209023. The deposited clone is contained in the pBluescript SK(−) plasmid (Stratagene, La Jolla, Calif.).

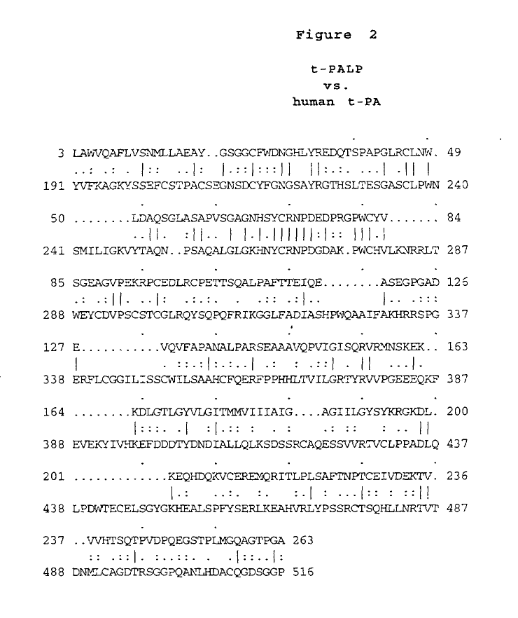

The t-PALP protein of the present invention shares sequence homology with the translation product of the human mRNA for t-PA (FIG. 2) (SEQ ID NO:3). t-PA is thought to be an important regulator of the dissolution of fibrin clots in humans and other animals. Abnormal blood clotting can lead to many vascular diseases, such as stroke, deep-vein thrombosis, peripheral arterial occlusion, pulmonary embolism, and myocardiothrombosis, each of which constitutes a major health risk. Such diseases are primarily caused by partial or total occlusion of a blood vessel by a blood clot. Such clots consist essentially of a mass of fibrin and platelets. The dissolution of existing clots is frequently achieved by the activation of plasminogen which dissolves the existing blood clot (thereby affecting the fibrinolysis pathway).

The fibrinolytic system is activated by the deposition of fibrin. t-PA activates plasminogen and, only upon activation, can plasminogen degrade fibrin, and, ultimately, degrade the blood clot itself.

Nucleic Acid Molecules

Unless otherwise indicated, all nucleotide sequences determined by sequencing a DNA molecule herein were determined using an automated DNA sequencer (such as the Model 373 from Applied Biosystems, Inc., Foster City, Calif.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule (these values may also be expressed as at most 10% different, more typically at most about 5% to about 0.1% different from the actual nucleotide sequence of the sequenced DNA molecule). The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Using the information provided herein, such as the nucleotide sequence in FIG. 1 (SEQ ID NO:1), a nucleic acid molecule of the present invention encoding a t-PALP polypeptide may be obtained using standard cloning and screening procedures, such as those for cloning cDNAs using mRNA as starting material. Illustrative of the invention, the nucleic acid molecule described in FIG. 1 (SEQ ID NO:1) was discovered in a cDNA library derived from activated monocytes.

Additional clones of the same gene were also identified in cDNA libraries from the following tissues: cerebellum, smooth muscle, resting and PHA-treated T-cells, GM-CSF-treated macrophages, frontal cortex of the brain, breast lymph node, chronic lymphocytic leukemic spleen, and several others.

A Northern blot analysis of the t-PALP clone of FIG. 1 (SEQ ID NO:1), or the t-PALP clone contained in ATCC Deposit No. 209023, indicated that 2.5 kb t-PALP message is detectable in heart, brain, placenta, lung, liver, skeletal muscle, kidney, pancreas, spleen, thymus, prostate, testis, ovary, small intestine, colon, and peripheral blood leukocytes (see Example 4).

The determined nucleotide sequence of the t-PALP cDNA of FIG. 1 (SEQ ID NO:1) contains an open reading frame encoding a protein of 263 amino acid residues, with an initiation codon at nucleotide positions 124-126 of the nucleotide sequence in FIG. 1 (SEQ ID NO:1), and a deduced molecular weight of about 28.2 kDa. An in vitro transcription/translation analysis of the t-PALP clone shown in SEQ ID NO:1, or the t-PALP clone contained in ATCC Deposit No. 209023, resulted in the production of a protein product of about 35 kDa. The amino acid sequence of the t-PALP protein shown in SEQ ID NO:2 is about 21.3% identical to human mRNA for t-PA (FIG. 2; Degen, S. J., Rajput, B., and Reich, E. (1986) J. Biol. Chem. 261:6972-6985; GenBank Accession No. K03021).

The open reading frame of the t-PALP gene shares sequence homology with the translation product of the human mRNA for t-PA (FIG. 2) (SEQ ID NO:3), including the following conserved domains: (a) the predicted kringle domain of about 59 amino acids, and (b) the predicted protease domain of about 179 amino acids. t-PA is thought to be important in the regulation of blood clotting and disorders related thereto. The homology between t-PA and t-PALP indicates that t-PALP may also be involved in the regulation of normal and abnormal clotting in such conditions including many vascular diseases, such as stroke, deep-vein thrombosis, peripheral arterial occlusion, pulmonary embolism, and myocardiothrombosis.

As one of ordinary skill would appreciate, due to the possibilities of sequencing errors discussed above, the actual complete t-PALP polypeptide encoded by the deposited cDNA, which comprises about 263 amino acids, may be somewhat longer or shorter. More generally, the actual open reading frame may be anywhere in the range of ±20 amino acids, more likely in the range of ±10 amino acids, of that predicted from the methionine codon at the N-terminus shown in FIG. 1 (SEQ ID NO:1). It will further be appreciated that, depending on the analytical criteria used for identifying various functional domains, the exact “address” of the kringle and protease domains of the t-PALP polypeptide may differ slightly from the predicted positions above. For example, the exact location of the t-PALP kringle and protease domains in SEQ ID NO:2 may vary slightly (e.g., the address may “shift” by about 1 to about 20 residues, more likely about 1 to about 5 residues) depending on the criteria used to define the domain.

Leader and Mature Sequences

The amino acid sequence of the complete t-PALP protein includes a leader sequence and a mature protein, as shown in SEQ ID NO:2. More in particular, the present invention provides nucleic acid molecules encoding a mature form of the t-PALP protein. Thus, according to the signal hypothesis, once export of the growing protein chain across the rough endoplasmic reticulum has been initiated, proteins secreted by mammalian cells have a signal or secretory leader sequence which is cleaved from the complete polypeptide to produce a secreted “mature” form of the protein. Most mammalian cells and even insect cells cleave secreted proteins with the same specificity. However, in some cases, cleavage of a secreted protein is not entirely uniform, which results in two or more mature species of the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature t-PALP polypeptide having the amino acid sequence encoded by the cDNA clone contained in the host identified as ATCC Deposit No. 209023. By the “mature t-PALP polypeptide having the amino acid sequence encoded by the cDNA clone in ATCC Deposit No. 209023” is meant the mature form(s) of the t-PALP protein produced by expression in a mammalian cell (e.g., COS cells, as described below) of the complete open reading frame encoded by the human DNA sequence of the clone contained in the vector in the deposited host.

In addition, methods for predicting whether a protein has a secretory leader as well as the cleavage point for that leader sequence are available. For instance, the method of McGeoch (Virus Res. 3:271-286 (1985)) uses the information from a short N-terminal charged region and a subsequent uncharged region of the complete (uncleaved) protein. The method of von Heinje (Nucleic Acids Res. 14:4683-4690 (1986)) uses the information from the residues surrounding the cleavage site, typically residues −13 to +2 where +1 indicates the amino terminus of the mature protein. The accuracy of predicting the cleavage points of known mammalian secretory proteins for each of these methods is in the range of 75-80% (von Heinje, supra). However, the two methods do not always produce the same predicted cleavage point(s) for a given protein.

In the present case, the deduced amino acid sequence of the complete t-PALP polypeptide was analyzed by a computer program PSORT, available from Dr. Kenta Nakai of the Institute for Chemical Research, Kyoto University (see K. Nakai and M. Kanehisa, Genomics 14:897-911 (1992)), which is an expert system for predicting the cellular location of a protein based on the amino acid sequence. As part of this computational prediction of localization, the methods of McGeoch and von Heinje are incorporated. Thus, the computation analysis described above predicted a single cleavable N-terminal signal sequence within the complete amino acid sequence shown in SEQ ID NO:2.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiation codon at positions 124-126 of the nucleotide sequence shown in FIG. 1 (SEQ ID NO:1).

Also included are DNA molecules comprising the coding sequence for the predicted mature t-PALP protein shown at positions 1-242 of SEQUENCE ID NO:2.

In addition, isolated nucleic acid molecules of the invention include DNA molecules which comprise a sequence substantially different from those described above but which, due to the degeneracy of the genetic code, still encode the t-PALP protein. Of course, the genetic code and species-specific codon preferences are well known in the art. Thus, it would be routine for one skilled in the art to generate the degenerate variants described above, for instance, to optimize codon expression for a particular host (e.g., change codons in the human mRNA to those preferred by a bacterial host such as E. coli).

In another aspect, the invention provides isolated nucleic acid molecules encoding the t-PALP polypeptide having an amino acid sequence encoded by the cDNA clone contained in the plasmid deposited as ATCC Deposit No. 209023 on May 8, 1997.

Preferably, this nucleic acid molecule will encode the mature polypeptide encoded by the above-described deposited cDNA clone.

The invention further provides an isolated nucleic acid molecule having the nucleotide sequence shown in FIG. 1 (SEQ ID NO:1) or the nucleotide sequence of the t-PALP cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the t-PALP gene in human tissue, for instance, by Northern blot analysis.

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NO:1 which consists of positions 1-915 of SEQ ID NO:1.

In addition, the invention provides nucleic acid molecules having nucleotide sequences related to extensive portions of SEQ ID NO:1 which have been determined from the following related cDNA clones: HTAAM28R (SEQ ID NO:4), HFKBA12R (SEQ ID NO:5), HAPBL24R (SEQ ID NO:6), HLMFG34R (SEQ ID NO:7), HHPGT42R (SEQ ID NO:8), HSSAX27R (SEQ ID NO:9), and HSSES93R (SEQ ID NO:10).

Further, the invention includes a polynucleotide comprising any portion of at least about 30 nucleotides, preferably at least about 50 nucleotides, of SEQ ID NO:1 from residue 1 to 110 and from 630 to 750. More preferably, the invention includes a polynucleotide comprising nucleotide residues 1 to 2000, 1 to 1500, 1 to 1000, 1 to 500, 1 to 250, 250 to 2000, 250 to 1500, 250 to 1000, 250 to 500, 500 to 2000, 500 to 1500, 500 to 1000, 1000 to 2000, and 1000 to 1500.

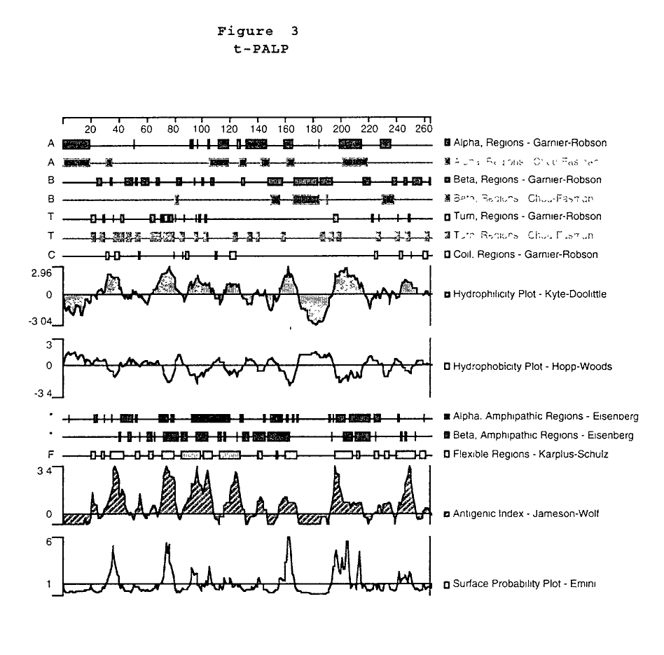

More generally, by a fragment of an isolated nucleic acid molecule having the nucleotide sequence of the deposited cDNA or the nucleotide sequence shown in FIG. 1 (SEQ ID NO:1) is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50-300 nt in length are also useful according to the present invention as are fragments corresponding to most, if not all, of the nucleotide sequence of the deposited cDNA or as shown in FIG. 1 (SEQ ID NO:1). By a fragment at least 20 nt in length, for example, is intended fragments which include 20 or more contiguous bases from the nucleotide sequence of the deposited cDNA or the nucleotide sequence as shown in FIG. 1 (SEQ ID NO:1). Preferred nucleic acid fragments of the present invention include nucleic acid molecules encoding epitope-bearing portions of the t-PALP polypeptide as identified in FIG. 3 and described in more detail below.

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit No. 209023. By “stringent hybridization conditions” is intended overnight incubation at 42° C. in a solution comprising: 50% formamide, 5× SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 (e.g., 50) nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIG. 1 (SEQ ID NO:1)). Of course, a polynucleotide which hybridizes only to a poly A sequence (such as the 3′ terminal poly(A) tract of the t-PALP cDNA shown in FIG. 1 (SEQ ID NO:1)), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode a t-PALP polypeptide may include, but are not limited to those encoding the amino acid sequence of the mature polypeptide, by itself; and the coding sequence for the mature polypeptide and additional sequences, such as those encoding the about 21 amino acid leader or secretory sequence, such as a pre-, or pro- or prepro-protein sequence; the coding sequence of the mature polypeptide, with or without the aforementioned additional coding sequences.

Also encoded by nucleic acids of the invention are the above protein sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the t-PALP fused to Fc at the N- or C-terminus.

Variant and Mutant Polynucleotides

The present invention further relates to variants of the nucleic acid molecules of the present invention, which encode portions, analogs or derivatives of the t-PALP protein. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the t-PALP protein or portions thereof. Also especially preferred in this regard are conservative substitutions.

Most highly preferred are nucleic acid molecules encoding the mature protein having the amino acid sequence shown in SEQ ID NO:2 or the mature t-PALP amino acid sequence encoded by the deposited cDNA clone.

Most highly preferred are nucleic acid molecules encoding the protease domain of the protein having the amino acid sequence shown in SEQ ID NO:2 or the protease domain of the t-PALP amino acid sequence encoded by the deposited cDNA clone.

Thus, one aspect of the invention provides an isolated nucleic acid molecule comprising a polynucleotide having a nucleotide sequence selected from the group consisting of: (a) a nucleotide sequence encoding a full-length t-PALP polypeptide having the complete amino acid sequence in SEQ ID NO:2 excepting the N-terminal methionine (i.e., positions −20 to 242 of SEQ ID NO:2) or the complete amino acid sequence excepting the N-terminal methionine encoded by the cDNA clone contained in the ATCC Deposit No. 209023; (b) a nucleotide sequence encoding the predicted mature form of the t-PALP polypeptide having the amino acid sequence at positions 1 to 242 in SEQ ID NO:2 or as encoded by the cDNA clone contained in the ATCC Deposit No. 209023; (c) a nucleotide sequence encoding the predicted kringle domain of the t-PALP polypeptide having the amino acid sequence at positions 4 to 63 in SEQ ID NO:2 or as encoded by the cDNA clone contained in the ATCC Deposit No. 209023; (d) a nucleotide sequence encoding a polypeptide comprising the predicted protease domain of the t-PALP polypeptide having the amino acid sequence at positions 64 to 242 in SEQ ID NO:2 or as encoded by the cDNA clone contained in the ATCC Deposit No. 209023; and (e) a nucleotide sequence complementary to any of the nucleotide sequences in (a), (b), (c), (d) or (e) above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d) or (e) above, or a polynucleotide which hybridizes under stringent hybridization conditions to a polynucleotide in (a), (b), (c), (d) or (e) above. In other words, these embodiments of the invention include isolated nucleic acid molecules that comprise a polynucleotide having a nucleotide sequence which contains at most 10% differences, and more preferably, at most 5%, 4%, 3%, 2% or 1% differences, with any of the nucleotide sequences in (a), (b), (c), (d) or (e) above, or a polynucleotide which hybridizes under stringent hybridization conditions to a polynucleotide in (a), (b), (c), (d) or (e) above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a t-PALP polypeptide having an amino acid sequence in (a), (b), (c) or (d) above.

The present invention also relates to recombinant vectors, which include the isolated nucleic acid molecules of the present invention, and to host cells containing the recombinant vectors, as well as to methods of making such vectors and host cells and for using them for production of t-PALP polypeptides or peptides by recombinant techniques.

By a polynucleotide having a nucleotide sequence at least, for example, 95% “identical” (that is, having 5% differences) to a reference nucleotide sequence encoding a t-PALP polypeptide is intended that the nucleotide sequence of the polynucleotide is identical to the reference sequence except that the polynucleotide sequence may include up to five point mutations per each 100 nucleotides of the reference nucleotide sequence encoding the t-PALP polypeptide. In other words, to obtain a polynucleotide having a nucleotide sequence at least 95% identical to a reference nucleotide sequence, up to 5% of the nucleotides in the reference sequence may be deleted, inserted or substituted with another nucleotide. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to (or 10%, 5%, 4%, 3%, 2% or 1% different from), for instance, the nucleotide sequence shown in FIG. 1 or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to (or 5% different from) a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 90%, 95%, 96%, 97%, 98% or 99% identical to (or stated in another way, at most 10%, 5%, 4%, 3%, 2% or 1% different from) the nucleic acid sequence shown in FIG. 1 (SEQ ID NO:1) or to the nucleic acid sequence of the deposited cDNA, irrespective of whether they encode a polypeptide having t-PALP activity. This is because even where a particular nucleic acid molecule does not encode a polypeptide having t-PALP activity, one of skill in the art would still know how to use the nucleic acid molecule, for instance, as a hybridization probe or a polymerase chain reaction (PCR) primer. Uses of the nucleic acid molecules of the present invention that do not encode a polypeptide having t-PALP activity include, inter alia, (1) isolating the t-PALP gene or allelic variants thereof in a cDNA library; (2) in situ hybridization (e.g., “FISH”) to metaphase chromosomal spreads to provide precise chromosomal location of the t-PALP gene, as described in Verma et al., Human Chromosomes: A Manual of Basic Techniques, Pergamon Press, New York (1988); and Northern Blot analysis for detecting t-PALP mRNA expression in specific tissues.

Preferred, however, are nucleic acid molecules having sequences at least 90%, 95%, 96%, 97%, 98% or 99% identical to (or 10%, 5%, 4%, 3%, 2% or 1% different from) the nucleic acid sequence shown in FIG. 1 (SEQ ID NO:1) or to the nucleic acid sequence of the deposited cDNA which do, in fact, encode a polypeptide having t-PALP protein activity. By “a polypeptide having t-PALP activity” is intended polypeptides exhibiting activity similar, but not necessarily identical, to an activity of the mature t-PALP protein of the invention, as measured in a particular biological assay. For example, the t-PALP protein of the present invention binds to fibrin. Such binding is assumed to mediate the stimulation of plasminogen activation and the ultimate lysis of a plasma clot. The ability of t-PALP, or other related proteins, to bind to fibrin may be assayed in an in vitro analysis, as described by Kalyan and colleagues (J. Biol. Chem. 263:3971-3978; 1988). Briefly, a fibrin clot is generated by clotting fibrinogen by the addition of thrombin to 1 unit/mL, incubating for 1 h at room temperature, and compacting by centrifugation. The clot is then washed once with 50 mM Tris-HCl (pH 7.4), 38 mM NaCl. Approximately 1000-2000 ng/mL of isolated t-PALP, or another related protein, are then incubated with the above-described plasminogen-free fibrin clot in a binding buffer consisting of 50 mM Tris-HCl (pH 7.4), 38 mM NaCl, 100 mg/mL albumin, 1600 mg/mL (˜5 mM) fibrinogen (plasminogen-free) for 1 h at room temperature. Again, the clot is compacted by centrifugation and washed once with 50 mM Tris-HCl (pH 7.4), 38 mM NaCl. The binding of t-PALP, or other related protein, to fibrin is then quantitated by gel elcetrophoresis and fibrin autography. Such fibrin-binding activity is a useful means of quantifying the ability of t-PALP, or a related protein, to bind to fibrin.

In addition, a general amidolytic activity of t-PALP, or another related protein, may also be assessed through the use of a simple biochemical assay also described by Kalyan and colleagues (J. Biol. Chem. 263:3971-3978; 1988). Cleavage of a synthetic chromogenic substrate (S-2288) may be used to assess the general amidolytic activity of t-PALP, or another related protein. Hydrolysis of this compound produces p-nitroaniline which may be easily quantitated spectrophotometrically by its absorbance at 405 nm. Amidolytic reactions contain 150 mM Tris-HCl (ph 8.4), 100 mg/mL albumin, 0.01% Tween-80, 4 nM t-PALP, or other related protein, and 0.6 mM S-2288. Reactions are performed in in microtiter plates and the differential absorbance at 405-540 nm are recorded at ten minute intervals up to 1 hour. Results are plotted as absorbance versus time. This analysis can be enhanced with a slight alteration.

Since it is well-known that fibrin greatly enhances plasminogen activation by t-PA and t-PALP, the generation of plasmin so formed can by conveniently measured by the slightly modified amidolytic assay. In this assay, the chromogenic substrate used is S-2251 (D-Val-L-Ile-Lys-p-nitroanalide). Plasminogen activation reactions contain 50 mM Tris-HCl (ph 7.4), 150 mM NaCl, 100 mg/mL albumin, 0.01% Tween-80, 0.3 nM t-PALP, or other related protein, 0.6 mM S-2251, 125 mg/mL soluble fibrin, and 1.5 mg/mL Glu-plasminogen. Reactions are performed in microtiter plates and are initiated by the addition of plasminogen and S-2251. The differential absorbance at 405-540 nm is recorded at 15 minute intervals and plotted as absorbance versus time.

Further, the activity of t-PALP, or another related polypeptide, can be assessed by using a plasma clot lysis assay, essentially as described Kalyan and colleagues (J. Biol. Chem. 263:3971-3978; 1988). In this analysis, the ability of t-PALP, or another related polypeptide, to lyse radiolabeled preformed plasma clots are assessed by bathing clots in plasma containing an appropriate concentration of t-PALP, or another related polypeptide, and monitoring the release of degraded, radiolabeled fibrin. In this assay, 100 mL of human citrated plasma is clotted in the presence of 0.5 mCi ¹²⁵I-fibrinogen by the addition of CaCl₂ to 25 mM and 2 units/mL thrombin. The clot is allowed to form at room temperature for 24 hours. The radioactively-labeled clot is then bathed in 1 mL of plasma which contains a series of concentrations of t-PALP, or another related polypeptide, (12.5 to 200 ng/mL). The reactions are shaken gently at 37° C. and samples are taken from the reactions at timepoints up to 24 hours. Aliquots of each sample (10 mL) are counted in a g counter and expressed as the percent of total counts expected from complete clot lysis.

t-PALP protein binds fibrin, has amidolytic activity, and can lyse a plasma clot in a dose-dependent manner in the above-described assays. Thus, “a polypeptide having t-PALP protein activity” includes polypeptides that also exhibit any of the same activities in the above-described assays in a dose-dependent manner. Although the degree of dose-dependent activity need not be identical to that of the t-PALP protein, preferably, “a polypeptide having t-PALP protein activity” will exhibit substantially similar dose-dependence in a given activity as compared to the t-PALP protein (i.e., the candidate polypeptide will exhibit greater activity or not more than about 25-fold less and, preferably, not more than about tenfold less activity relative to the reference t-PALP protein).

Of course, due to the degeneracy of the genetic code, one of ordinary skill in the art will immediately recognize that a large number of the nucleic acid molecules having a sequence at least 90%, 95%, 96%, 97%, 98%, or 99% identical to (or 10%, 5%, 4%, 3%, 2% or 1% different from) the nucleic acid sequence of the deposited cDNA or the nucleic acid sequence shown in FIG. 1 (SEQ ID NO:1) will encode a polypeptide “having t-PALP protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having t-PALP protein activity. This is because the skilled artisan is fully aware of amino acid substitutions that are either less likely or not likely to significantly effect protein function (e.g., replacing one aliphatic amino acid with a second aliphatic amino acid), as further described below.

Vectors and Host Cells

The present invention also relates to vectors which include the isolated DNA molecules of the present invention, host cells which are genetically engineered with the recombinant vectors, and the production of t-PALP polypeptides or fragments thereof by recombinant techniques. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc., supra; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5, have been fused with Fc portions for the purpose of high-throughput screening assays to identify antagonists of hIL-5. See, D. Bennett et al., J. Molecular Recognition 8:52-58 (1995) and K. Johanson et al., J. Biol. Chem. 270:9459-9471 (1995).

The t-PALP protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Polypeptides and Fragments

The invention further provides an isolated t-PALP polypeptide having the amino acid sequence encoded by the deposited cDNA, or the amino acid sequence in SEQ ID NO:2, or a peptide or polypeptide comprising a portion of the above polypeptides.

Variant and Mutant Polypeptides

To improve or alter the characteristics of t-PALP polypeptides, protein engineering may be employed. Recombinant DNA technology known to those skilled in the art can be used to create novel mutant proteins or “muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

N-terminal and C-terminal Deletion Mutants

For instance, for many proteins, including the extracellular domain of a membrane associated protein or the mature form(s) of a secreted protein, it is known in the art that one or more amino acids may be deleted from the N-terminus or C-terminus without substantial loss of biological function. For instance, Ron and colleagues (J. Biol. Chem., 268:2984-2988; 1993) reported modified KGF proteins that had heparin binding activity even if 3, 8, or 27 amino-terminal amino acid residues were missing. In the present case, since the protein of the invention is related to t-PA, deletions of N-terminal amino acids up to the serine at position 64 of SEQ ID NO:2 may retain some proteolytic activity. Polypeptides having further N-terminal deletions including the serine residue in SEQ ID NO:2 would not be expected to retain such biological activities because it is known that this residue in t-PA is in the beginning of the conserved protease domain required for its observed proteolytic activity.

However, even if deletion of one or more amino acids from the N-terminus of a protein results in modification of loss of one or more biological functions of the protein, other biological activities may still be retained. Thus, the ability of the shortened protein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of the t-PALP shown in SEQ ID NO:2, up to the serine residue at position number 64, and polynucleotides encoding such polypeptides. In particular, the present invention provides polypeptides comprising the amino acid sequence of residues n-242 of SEQ ID NO:2, where n is an integer in the range of −21-64, and 64 is the position of the first residue from the N-terminus of the complete t-PALP polypeptide (shown in SEQ ID NO:2) believed to be required for proteolytic activity of the t-PALP protein.

More in particular, the invention provides polynucleotides encoding polypeptides having the amino acid sequence of residues of −20-242, −19-242, −18-242, −17-242, −16-242, −15-242, −14-242, −13-242, −12-242, −11-242, −10-242, −9-242, −8-242, −7-242, −6-242, −5-242, −4-242, −3-242, −2-242, −1-242, 1-242, 2-242, 3-242, 4-242, 5-242, 6-242, 7-242, 8-242, 9-242, 10-242, 11-242, 12-242, 13-242, 14-242, 15-242, 16-242, 17-242, 18-242, 19-242, 20-242, 21-242, 22-242, 23-242, 24-242, 25-242, 26-242, 27-242, 28-242, 29-242, 30-242, 31-242, 32-242, 33-242, 34-242, 35-242, 36-242, 37-242, 38-242, 39-242, 40-242, 41-242, 42-242, 43-242, 44-242, 45-242, 46-242, 47-242, 48-242, 49-242, 50-242, 51-242, 52-242, 53-242, 54-242, 55-242, 56-242, 57-242, 58-242, 59-242, 60-242, 61-242, 62-242, 63-242, of SEQ ID NO:2. Polynucleotides encoding these polypeptides also are provided.

Similarly, many examples of biologically functional C-terminal deletion muteins are known. For instance, Interferon-g shows up to ten times higher activities by deleting 8-10 amino acid residues from the carboxy terminus of the protein (Dobeli et al., (1988) J. Biotechnol. 7:199-216). In the present case, since the protein of the invention is a member of the serine protease or t-PA polypeptide families, deletions of C-terminal amino acids up to the serine at position 230 of SEQ ID NO:2 may retain some of the observed proteolytic activity of the carboxy-terminal protease domain of t-PA.

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues from the carboxy terminus of the amino acid sequence of the t-PALP shown in SEQ ID NO:2, up to the serine residue at position 230 of SEQ ID NO:2, and polynucleotides encoding such polypeptides. In particular, the present invention provides polypeptides having the amino acid sequence of residues −20-m of the amino acid sequence in SEQ ID NO:2, where m is any integer in the range of 230 to 241, and residue serine is the position of the first residue from the C-terminus of the complete t-PALP polypeptide (shown in SEQ ID NO:2) believed to be required for protease of the t-PALP protein.

More in particular, the invention provides polynucleotides encoding polypeptides having the amino acid sequence of residues −20-230, −20-231, −20-232, −20-233, −20-234, −20-235, −20-236, −20-237, −20-238, −20-239, −20-240, −20-241, −20-242 of SEQ ID NO:2. Polynucleotides encoding these polypeptides also are provided.

The invention also provides polypeptides having one or more amino acids deleted from both the amino and the carboxyl termini, which may be described generally as having residues n-m of SEQ ID NO:2, where n and m are integers as described above.

Also included are a nucleotide sequence encoding a polypeptide consisting of a portion of the complete t-PALP amino acid sequence encoded by the cDNA clone contained in ATCC Deposit No. 209023, where this portion excludes from 1 to about 63 amino acids from the amino terminus of the complete amino acid sequence encoded by the cDNA clone contained in ATCC Deposit No. 209023, or from 1 to about 11 amino acids from the carboxy terminus, or any combination of the above amino terminal and carboxy terminal deletions, of the complete amino acid sequence encoded by the cDNA clone contained in ATCC Deposit No. 209023. Polynucleotides encoding all of the above deletion mutant polypeptide forms also are provided.

As mentioned above, even if deletion of one or more amino acids from the N-terminus of a protein results in modification of loss of one or more biological functions of the protein, other biological activities may still be retained. Thus, the ability of the shortened t-PALP mutein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art. It is not unlikely that a t-PALP mutein with a large number of deleted N-terminal amino acid residues may retain some biological or immungenic activities. In fact, peptides composed of as few as six t-PALP amino acid residues may often evoke an immune response.

Accordingly, the present invention further provides polypeptides having one or more residues deleted from the amino terminus of the amino acid sequence of the t-PALP shown in SEQ ID NO:2, up to the alanine residue at position number 258 (numbering as shown in FIG. 1; A-258 is A-237 in SEQ ID NO:2), and polynucleotides encoding such polypeptides. In particular, the present invention provides polypeptides comprising the amino acid sequence of residues n′-258 of FIG. 1 (n′-237 of SEQ ID NO:2), where n′ is an integer in the range of 2-258 (−21-258 of SEQ ID NO:2), and 258 is the position of the first residue from the N-terminus of the complete t-PALP polypeptide (shown as residue 237 in SEQ ID NO:2) believed to be required for at least immunogenic activity of the t-PALP protein.

More in particular, the invention provides polynucleotides encoding polypeptides having the amino acid sequence of residues of L-2 to A-263; L-3 to A-263; A-4 to A-263; W-5 to A-263; V-6 to A-263; Q-7 to A-263; A-8 to A-263; F-9 to A-263; L-10 to A-263; V-11 to A-263; S-12 to A-263; N-13 to A-263; M-14 to A-263; L-15 to A-263; L-16 to A-263; A-17 to A-263; E-18 to A-263; A-19 to A-263; Y-20 to A-263; G-21 to A-263; S-22 to A-263; G-23 to A-263; G-24 to A-263; C-25 to A-263; F-26 to A-263; W-27 to A-263; D-28 to A-263; N-29 to A-263; G-30 to A-263; H-31 to A-263; L-32 to A-263; Y-33 to A-263; R-34 to A-263; E-35 to A-263; D-36 to A-263; Q-37 to A-263; T-38 to A-263; S-39 to A-263; P-40 to A-263; A-41 to A-263; P-42 to A-263; G-43 to A-263; L-44 to A-263; R-45 to A-263; C-46 to A-263; L-47 to A-263; N-48 to A-263; W-49 to A-263; L-50 to A-263; D-51 to A-263; A-52 to A-263; Q-53 to A-263; S-54 to A-263; G-55 to A-263; L-56 to A-263; A-57 to A-263; S-58 to A-263; A-59 to A-263; P-60 to A-263; V-61 to A-263; S-62 to A-263; G-63 to A-263; A-64 to A-263; G-65 to A-263; N-66 to A-263; H-67 to A-263; S-68 to A-263; Y-69 to A-263; C-70 to A-263; R-71 to A-263; N-72 to A-263; P-73 to A-263; D-74 to A-263; E-75 to A-263; D-76 to A-263; P-77 to A-263; R-78 to A-263; G-79 to A-263; P-80 to A-263; W-81 to A-263; C-82 to A-263; Y-83 to A-263; V-84 to A-263; S-85 to A-263; G-86 to A-263; E-87 to A-263; A-88 to A-263; G-89 to A-263; V-90 to A-263; P-91 to A-263; E-92 to A-263; K-93 to A-263; R-94 to A-263; P-95 to A-263; C-96 to A-263; E-97 to A-263; D-98 to A-263; L-99 to A-263; R-100 to A-263; C-101 to A-263; P-102 to A-263; E-103 to A-263; T-104 to A-263; T-105 to A-263; S-106 to A-263; Q-107 to A-263; A-108 to A-263; L-109 to A-263; P-110 to A-263; A-111 to A-263; F-112 to A-263; T-113 to A-263; T-114 to A-263; E-115 to A-263; I-116 to A-263; Q-117 to A-263; E-118 to A-263; A-119 to A-263; S-120 to A-263; E-121 to A-263; G-122 to A-263; P-123 to A-263; G-124 to A-263; A-125 to A-263; D-126 to A-263; E-127 to A-263; V-128 to A-263; Q-129 to A-263; V-130 to A-263; F-131 to A-263; A-132 to A-263; P-133 to A-263; A-134 to A-263; N-135 to A-263; A-136 to A-263; L-137 to A-263; P-138 to A-263; A-139 to A-263; R-140 to A-263; S-141 to A-263; E-142 to A-263; A-143 to A-263; A-144 to A-263; A-145 to A-263; V-146 to A-263; Q-147 to A-263; P-148 to A-263; V-149 to A-263; I-150 to A-263; G-151 to A-263; I-152 to A-263; S-153 to A-263; Q-154 to A-263; R-155 to A-263; V-156 to A-263; R-157 to A-263; M-158 to A-263; N-159 to A-263; S-160 to A-263; K-161 to A-263; E-162 to A-263; K-163 to A-263; K-164 to A-263; D-165 to A-263; L-166 to A-263; G-167 to A-263; T-168 to A-263; L-169 to A-263; G-170 to A-263; Y-171 to A-263; V-172 to A-263; L-173 to A-263; G-174 to A-263; I-175 to A-263; T-176 to A-263; M-177 to A-263; M-178 to A-263; V-179 to A-263; I-180 to A-263; I-181 to A-263; I-182 to A-263; A-183 to A-263; I-184 to A-263; G-185 to A-263; A-186 to A-263; G-187 to A-263; I-188 to A-263; I-189 to A-263; L-190 to A-263; G-191 to A-263; Y-192 to A-263; S-193 to A-263; Y-194 to A-263; K-195 to A-263; R-196 to A-263; G-197 to A-263; K-198 to A-263; D-199 to A-263; L-200 to A-263; K-201 to A-263; E-202 to A-263; Q-203 to A-263; H-204 to A-263; D-205 to A-263; Q-206 to A-263; K-207 to A-263; V-208 to A-263; C-209 to A-263; E-210 to A-263; R-211 to A-263; E-212 to A-263; M-213 to A-263; Q-214 to A-263; R-215 to A-263; 1-216 to A-263; T-217 to A-263; L-218 to A-263; P-219 to A-263; L-220 to A-263; S-221 to A-263; A-222 to A-263; F-223 to A-263; T-224 to A-263; N-225 to A-263; P-226 to A-263; T -227 to A-263; C-228 to A-263; E-229 to A-263; I-230 to A-263; V-231 to A-263; D-232 to A-263; E-233 to A-263; K-234 to A-263; T-235 to A-263; V-236 to A-263; V-237 to A-263; V-238 to A-263; H-239 to A-263; T-240 to A-263; S-241 to A-263; Q-242 to A-263; T-243 to A-263; P-244 to A-263; V-245 to A-263; D-246 to A-263; P-247 to A-263; Q-248 to A-263; E-249 to A-263; G-250 to A-263; S-251 to A-263; T-252 to A-263; P-253 to A-263; L-254 to A-263; M-255 to A-263; G -256 to A-263; Q-257 to A-263; and A-258 to A-263 of the t-PALP sequence shown in SEQ ID NO:2 using the numbering scheme of FIG. 1.

Also as mentioned above, even if deletion of one or more amino acids from the C-terminus of a protein results in modification of loss of one or more biological functions of the protein, other biological activities may still be retained. Thus, the ability of the shortened t-PALP mutein to induce and/or bind to antibodies which recognize the complete or mature of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art. It is not unlikely that a t-PALP mutein with a large number of deleted C-terminal amino acid residues may retain some biological or immungenic activities. In fact, peptides composed of as few as six t-PALP amino acid residues may often evoke an immune response.

Accordingly, the present invention further provides polypeptides having one or more residues deleted from the carboxy terminus of the amino acid sequence of the t-PALP shown in SEQ ID NO:2, up to the valine residue at position number 6 (numbering as shown in FIG. 1; the valine at position 6 is the valine at position −14 in SEQ ID NO:2), and polynucleotides encoding such polypeptides. In particular, the present invention provides polypeptides comprising the amino acid sequence of residues 1-m′ of FIG. 1 (−21-m′ of SEQ ID NO:2), where m′ is an integer in the range of 7-263 (−13-242 of SEQ ID NO:2), and 6 is the position of the first residue from the C-terminus of the complete t-PALP polypeptide (shown as residue −14 in SEQ ID NO:2) believed to be required for at least immunogenic activity of the t-PALP protein.

More in particular, the invention provides polynucleotides encoding polypeptides having the amino acid sequence of residues M-1 to G-262; M-1 to P-261; M-1 to T-260; M-1 to G-259; M-1 to A-258; M-1 to Q-257; M-1 to G-256; M-1 to M-255; M-1 to L-254; M-1 to P-253; M-1 to T-252; M-1 to S-251; M-1 to G-250; M-1 to E-249; M-1 to Q-248; M-1 to P-247; M-1 to D-246; M-1 to V-245; M-1 to P-244; M-1 to T-243; M-1 to Q-242; M-1 to S-241; M-1 to T-240; M-1 to H-239; M-1 to V-238; M-1 to V-237; M-1 to V-236; M-1 to T-235; M-1 to K-234; M-1 to E-233; M-1 to D-232; M-1 to V-231; M-1 to 1-230; M-1 to E-229; M-1 to C-228; M-1 to T-227; M-1 to P-226; M-1 to N-225; M-1 to T-224; M-1 to F-223; M-1 to A-222; M-1 to S-221; M-1 to L-220; M-1 to P-219; M-1 to L-218; M-1 to T-217; M-1 to I-216; M-1 to R-215; M-1 to Q-214; M-1 to M-213; M-1 to E-212; M-1 to R-211; M-1 to E-210; M-1 to C-209; M-1 to V-208; M-1 to K-207; M-1 to Q-206; M-1 to D-205; M-1 to H-204; M-1 to Q-203; M-1 to E-202; M-1 to K-201; M-1 to L-200; M-1 to D-199; M-1 to K-198; M-1 to G-197; M-1 to R-196; M-1 to K-195; M-1 to Y-194; M-1 to S-193; M-1 to Y-192; M-1 to G-191; M-1 to L-190; M-1 to I-189; M-1 to I-188; M-1 to G-187; M-1 to A-186; M-1 to G-185; M-1 to 1-184; M-1 to A-183; M-1 to I-182; M-1 to I-181; M-1 to I-180; M-1 to V-179; M-1 to M-178; M-1 to M-177; M-1 to T-176; M-1 to I-175; M-1 to G-174; M-1 to L-173; M-1 to V-172; M-1 to Y-171; M-1 to G-170; M-1 to L-169; M-1 to T-168; M-1 to G-167; M-1 to L-166; M-1 to D-165; M-1 to K-164; M-1 to K-163; M-1 to E-162; M-1 to K-161; M-1 to S-160; M-1 to N-159; M-1 to M-158; M-1 to R-157; M-1 to V-156; M-1 to R-155; M-1 to Q-154; M-1 to S-153; M-1 to I-152; M-1 to G-151; M-1 to I-150; M-1 to V-149; M-1 to P-148; M-1 to Q-147; M-1 to V-146; M-1 to A-145; M-1 to A-144; M-1 to A-143; M-1 to E-142; M-1 to S-141; M-1 to R-140; M-1 to A-139; M-1 to P-138; M-1 to L-137; M-1 to A-136; M-1 to N-135; M-1 to A-134; M-1 to P-133; M-1 to A-132; M-1 to F-131; M-1 to V-130; M-1 to Q-129; M-1 to V-128; M-1 to E-127; M-1 to D-126; M-1 to A-125; M-1 to G-124; M-1 to P-123; M-1 to G-122; M-1 to E-121; M-1 to S-120; M-1 to A-119; M-1 to E-118; M-1 to Q-117; M-1 to I-116; M-1 to E-115; M-1 to T-114; M-1 to T-113; M-1 to F-112; M-1 to A-111; M-1 to P-110; M-1 to L-109; M-1 to A-108; M-1 to Q-107; M-1 to S-106; M-1 to T-105; M-1 to T-104; M-1 to E-103; M-1 to P-102; M-1 to C-101; M-1 to R-100; M-1 to L-99; M-1 to D-98; M-1 to E-97; M-1 to C-96; M-1 to P-95; M-1 to R-94; M-1 to K-93; M-1 to E-92; M-1 to P-91; M-1 to V-90; M-1 to G-89; M-1 to A-88; M-1 to E-87; M-1 to G-86; M-1 to S-85; M-1 to V-84; M-1 to Y-83; M-1 to C-82; M-1 to W-81; M-1 to P-80; M-1 to G-79; M-1 to R-78; M-1 to P-77; M-1 to D-76; M-1 to E-75; M-1 to D-74; M-1 to P-73; M-1 to N-72; M-1 to R-71; M-1 to C-70; M-1 to Y-69; M-1 to S-68; M-1 to H-67; M-1 to N-66; M-1 to G-65; M-1 to A-64; M-1 to G-63; M-1 to S-62; M-1 to V-61; M-1 to P-60; M-1 to A-59; M-1 to S-58; M-1 to A-57; M-1 to L-56; M-1 to G-55; M-1 to S-54; M-1 to Q-53; M-1 to A-52; M-1 to D-51; M-1 to L-50; M-1 to W49; M-1 to N-48; M-1 to L-47; M-1 to C-46; M-1 to R-45; M-1 to L-44; M-1 to G-43; M-1 to P-42; M-1 to A-41; M-1 to P-40; M-1 to S-39; M-1 to T-38; M-1 to Q-37; M-1 to D-36; M-1 to E-35; M-1 to R-34; M-1 to Y-33; M-1 to L-32; M-1 to H-31; M-1 to G-30; M-1 to N-29; M-1 to D-28; M-1 to W-27; M-1 to F-26; M-1 to C-25; M-1 to G-24; M-1 to G-23; M-1 to S-22; M-1 to G-21; M-1 to Y-20; M-1 to A-19; M-1 to E-18; M-1 to A-17; M-1 to L-16; M-1 to L-15; M-1 to M-14; M-1 to N-13; M-1 to S-12; M-1 to V-11; M-1 to L-10; M-1 to F-9; M-1 to A-8; M-1 to Q-7; and M-1 to V-6 of the t-PALP sequence shown in SEQ ID NO:2 using the numbering scheme of FIG. 1. Polynucleotides encoding these polypeptides also are provided.

The invention also provides polypeptides having one or more amino acids deleted from both the amino and the carboxyl termini, which may be described generally as having residues n′-m′ of SEQ ID NO:2, where n′ and m′ are integers as described above.

Other Mutants

In addition to terminal deletion forms of the protein discussed above, it also will be recognized by one of ordinary skill in the art that some amino acid sequences of the t-PALP polypeptide can be varied without significant effect of the structure or function of the protein. If such differences in sequence are contemplated, it should be remembered that there will be critical areas on the protein which determine activity.

Thus, the invention further includes variations of the t-PALP polypeptide which show substantial t-PALP polypeptide activity or which include regions of t-PALP protein such as the protein portions discussed below. Such mutants include deletions, insertions, inversions, repeats, and type substitutions selected according to general rules known in the art so as have little effect on activity. For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, J. U. et al., supra, and the references cited therein. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Thus, the fragment, derivative or analog of the polypeptide of SEQ ID NO:2, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein Thus, the t-PALP of the present invention may include one or more amino acid substitutions, deletions or additions, either from natural mutations or human manipulation. As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Amino acids in the t-PALP protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).

A number of mutagenesis studies have been performed on the related t-PA polypeptide. The t-PA fibrin-binding activity has been mapped to the amino-terminal finger and EGF domains (Kalyan, N. K., et al., J. Biol. Chem. 263:3971-3978; 1988). In addition, in vivo clearance rates have also been mapped to the finger domain of t-PA (Ahern, T. J., et al., J. Biol. Chem. 265:5540-5545; 1990) Other studies by Yahara and colleagues (Thromb. and Haem. 72(6):893-899; 1994) report an in vivo clearance activity to be located not only in the finger domain, but also in the kringle domain of t-PA. Several mutations were identified in the protease domain which affected t-PA protease activity (Paoni, N. F., et al., Prot. Eng. 5:259-266; 1992). Fibrinolytic activity of t-PA was found to be reduced by mutation of one or more highly conserved amino acid residues in the kringle domains (Markland, W., et al., Prot. Eng. 3:117-125; 1989). A key study published by Haigwood and colleagues (Prot. Eng. 2:611-620; 1989) provided a detailed analysis of the effects of various insertion, deletion, and substitution mutations on the various activities of the t-PA molecule. The study determined that (1) variants with carbohydrate-depleted kringle domains possessed higher specific activities than wild-type t-PA, (2) a cleavage site variant substituted at Arg275 with Gly had greatly reduced specific activity, (3) two variants substituted at Lys277 exhibited altered interactions with plasminogen activator inhibitor (PAI)-2, (4) the variant with a truncated carboxy-terminus had reduced activity in the absence of fibrin, and (5) no variants had significantly altered half-lives. A molecular biologist skilled in the techniques of protein mutagenesis would infer from these and other studies that, since the various activities of t-PA may be altered by the introduction of various mutations into the molecule, that, by inference, it may be possible to also target specific mutations to the t-PALP molecule in an effort to reproduce similar changes in t-PALP activities. Since t-PALP is a member of the t-PA-related protein family, to modulate rather than completely eliminate biological activities of t-PALP, preferably mutations are made in sequences encoding amino acids in the t-PALP conserved kringle domain, i.e., in positions 4 to 63 of SEQ ID NO:2, more preferably in residues within this region which are not conserved in all members of the t-PA-related protein family. Similarly, preferable mutations are made in sequences encoding amino acids in the t-PALP conserved protease domain, i.e., in positions 64 to 242 of SEQ ID NO:2, more preferably in residues within this region which are not conserved in all members of the t-PA-related protein family. Also forming part of the present invention are isolated polynucleotides comprising nucleic acid sequences which encode the above t-PALP mutants.

The polypeptides of the present invention are preferably provided in an isolated form, and preferably are substantially purified. A recombinantly produced version of the t-PALP polypeptide can be substantially purified by the one-step method described by Smith and Johnson (Gene 67:31-40; 1988). Polypeptides of the invention also can be purified from natural or recombinant sources using anti-t-PALP antibodies of the invention in methods which are well known in the art of protein purification.

The invention further provides an isolated t-PALP polypeptide comprising an amino acid sequence selected from the group consisting of: (a) the amino acid sequence of the full-length t-PALP polypeptide having the complete amino acid sequence shown in SEQ ID NO:2 excepting the N-terminal methionine (i.e., positions −20 to 242 of SEQ ID NO:2) or the complete amino acid sequence excepting the N-terminal methionine encoded by the cDNA clone contained in the ATCC Deposit No. 209023; (b) the amino acid sequence comprising the predicted mature form of the t-PALP polypeptide having the amino acid sequence at positions 1 to 242 in SEQ ID NO:2 or as encoded by the cDNA clone contained in the ATCC Deposit No. 209023; (c) the amino acid sequence comprising the predicted kringle domain of the t-PALP polypeptide having the amino acid sequence at positions 4 to 63 in SEQ ID NO:2 or as encoded by the cDNA clone contained in the ATCC Deposit No. 209023; and (d) the amino acid sequence comprising the predicted protease domain of the t-PALP polypeptide having the amino acid sequence at positions 64 to 242 in SEQ ID NO:2 or as encoded by the cDNA clone contained in the ATCC Deposit No. 209023. The polypeptides of the present invention also include polypeptides having an amino acid sequence at least 80% identical (or 20% different), more preferably at least 90% identical (or 10% different), and still more preferably 95%, 96%, 97%, 98% or 99% identical to (or 5%, 4%, 3%, 2% or 1% different from) those described in (a), (b), (c) or (d) above, as well as polypeptides having an amino acid sequence with at least 90% similarity, and more preferably at least 95% similarity, to those above.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by the deposited cDNA or to the polypeptide of SEQ ID NO:2, and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

By a polypeptide having an amino acid sequence at least, for example, 95% “identical” to a reference amino acid sequence of a t-PALP polypeptide is intended that the amino acid sequence of the polypeptide is identical to the reference sequence except that the polypeptide sequence may include up to five amino acid alterations per each 100 amino acids of the reference amino acid of the t-PALP polypeptide. In other words, to obtain a polypeptide having an amino acid sequence at least 95% identical to (or 5% different from) a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to (or 10%, 5%, 4%, 3%, 2% or 1% different from), for instance, the amino acid sequence shown in SEQ ID NO:2 or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to (or 5% different from) a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting t-PALP protein expression as described below or as agonists and antagonists capable of enhancing or inhibiting t-PALP protein function. Further, such polypeptides can be used in the yeast two-hybrid system to “capture” t-PALP protein binding proteins which are also candidate agonists and antagonists according to the present invention. The yeast two hybrid system is described in Fields and Song, Nature 340:245-246 (1989).

Epitope-bearing Portions

In another aspect, the invention provides a peptide or polypeptide comprising an epitope-bearing portion of a polypeptide of the invention. The epitope of this polypeptide portion is an immnunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983) “Antibodies that react with predetermined sites on proteins,” Science, 219:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Antigenic epitope-bearing peptides and polypeptides of the invention are therefore useful to raise antibodies, including monoclonal antibodies, that bind specifically to a polypeptide of the invention. See, for instance, Wilson et al., Cell 37:767-778 (1984) at 777.

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to generate t-PALP-specific antibodies include: a polypeptide comprising amino acid residues from about Ser-1 to about His-10 in SEQ ID NO:2; about Glu-14 to about Leu-23 in SEQ ID NO:2; about Arg-50 to about Trp-60 in SEQ ID NO:2; about Pro-70 to about Gln-86 in SEQ ID NO:2; about Ala-98 to about Val-107 in SEQ ID NO:2; about Leu-117 to about Gln-126 in SEQ ID NO:2; about Arg-134 to about Gly-146 in SEQ ID NO:2; about Ser-172 to about Gln-182 in SEQ ID NO:2; about Gln-185 to about Arg-194 in SEQ ID NO:2; about Thr-206 to about Val-216 in SEQ ID NO:2; and about Thr-222 to about Thr-231 in SEQ ID NO:2; These polypeptide fragments have been determined to bear antigenic epitopes of the t-PALP protein by the analysis of the Jameson-Wolf antigenic index, as shown in FIG. 3, above.

The epitope-bearing peptides and polypeptides of the invention may be produced by any conventional means. See, e.g., Houghten, R. A. (1985) “General method for the rapid solid-phase synthesis of large numbers of peptides: specificity of antigen-antibody interaction at the level of individual amino acids.” Proc. Natl. Acad. Sci. USA 82:5131-5135; this “Simultaneous Multiple Peptide Synthesis (SMPS)” process is further described in U.S. Pat. No. 4,631,211 to Houghten et al. (1986).

Epitope-bearing peptides and polypeptides of the invention are used to induce antibodies according to methods well known in the art. See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985). Immunogenic epitope-bearing peptides of the invention, i.e., those parts of a protein that elicit an antibody response when the whole protein is the immunogen, are identified according to methods known in the art. See, for instance, Geysen et al., supra. Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat No. 5,480,971 to Houghten, R. A. et al. (1996) on Peralkylated Oligopeptide Mixtures discloses linear C1-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Fusion Proteins

As one of skill in the art will appreciate, t-PALP polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker et al., Nature 331:84-86 (1988)). Fusion proteins that have a disulfide-linked dimeric structure due to the IgG part can also be more efficient in binding and neutralizing other molecules than the monomeric t-PALP protein or protein fragment alone (Fountoulakis et al., J. Biochem. 270:3958-3964 (1995)).

Antibodies

t-PALP-protein specific antibodies for use in the present invention can be raised against the intact t-PALP protein or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (such as, for example, Fab and F(ab′)2 fragments) which are capable of specifically binding to t-PALP protein. Fab and F(ab′)2 fragments lack the Fc fragment of intact antibody, clear more rapidly from the circulation, and may have less non-specific tissue binding of an intact antibody (Wahl et al., J. Nucl. Med. 24:316-325 (1983)). Thus, these fragments are preferred.

The antibodies of the present invention may be prepared by any of a variety of methods. For example, cells expressing the t-PALP protein or an antigenic fragment thereof can be administered to an animal in order to induce the production of sera containing polyclonal antibodies. In a preferred method, a preparation of t-PALP protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or t-PALP protein binding fragments thereof). Such monoclonal antibodies can be prepared using hybridoma technology (Kohler et al., Nature 256:495 (1975); Köhler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., (1981) pp. 563-681 ). In general, such procedures involve immunizing an animal (preferably a mouse) with a t-PALP protein antigen or, more preferably, with a t-PALP protein-expressing cell. Suitable cells can be recognized by their capacity to bind anti-t-PALP protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP2O), available from the American Type Culture Collection, Rockville, Maryland. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the t-PALP protein antigen.

Alternatively, additional antibodies capable of binding to the t-PALP protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, t-PALP-protein specific antibodies are used to immunize an animal, preferably a mouse. The splenocytes of such an animal are then used to produce hybridoma cells, and the hybridoma cells are screened to identify clones which produce an antibody whose ability to bind to the t-PALP protein-specific antibody can be blocked by the t-PALP protein antigen. Such antibodies comprise anti-idiotypic antibodies to the t-PALP protein-specific antibody and can be used to immunize an animal to induce formation of further t-PALP protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, t-PALP protein-binding fragments can be produced through the application of recombinant DNA technology or through synthetic chemistry.

For in vivo use of anti-t-PALP in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

Circulatory System-related Disorders

Diagnosis

The present inventors have discovered that t-PALP is expressed in activated monocytes. For a number of circulatory system-related disorders, substantially altered (increased or decreased) levels of t-PALP gene expression can be detected in circulatory system tissue or other cells or bodily fluids (e.g., sera, plasma, urine, synovial fluid or spinal fluid) taken from an individual having such a disorder, relative to a “standard” t-PALP gene expression level, that is, the t-PALP expression level in circulatory system tissues or bodily fluids from an individual not having the circulatory system disorder. Thus, the invention provides a diagnostic method useful during diagnosis of a circulatory system disorder, which involves measuring the expression level of the gene encoding the t-PALP protein in circulatory system tissue or other cells or body fluid from an individual and comparing the measured gene expression level with a standard t-PALP gene expression level, whereby an increase or decrease in the gene expression level compared to the standard is indicative of an circulatory system disorder.

In particular, it is believed that certain tissues in mammals with cancers of the circulatory system express significantly reduced levels of the t-PALP protein and mRNA encoding the t-PALP protein when compared to a corresponding “standard” level. Further, it is believed that altered levels of the t-PALP protein can be detected in certain body fluids (e.g., sera, plasma, urine, and spinal fluid) from mammals with such a cancer when compared to sera from mammals of the same species not having the cancer.

Thus, the invention provides a diagnostic method useful during diagnosis of a circulatory system disorder, including cancers of this system, which involves measuring the expression level of the gene encoding the t-PALP protein in the circulatory system tissue or other cells or body fluid from an individual and comparing the measured gene expression level with a standard t-PALP gene expression level, whereby an increase or decrease in the gene expression level compared to the standard is indicative of a circulatory system disorder.

Where a diagnosis of a disorder in the circulatory system including diagnosis of a cancer has already been made according to conventional methods, the present invention is useful as a prognostic indicator, whereby patients exhibiting enhanced or depressed t-PALP gene expression will experience a worse clinical outcome relative to patients expressing the gene at a level nearer the standard level.

By “assaying the expression level of the gene encoding the t-PALP protein” is intended qualitatively or quantitatively measuring or estimating the level of the t-PALP protein or the level of the mRNA encoding the t-PALP protein in a first biological sample either directly (e.g., by determining or estimating absolute protein level or mRNA level) or relatively (e.g., by comparing to the t-PALP protein level or mRNA level in a second biological sample). Preferably, the t-PALP protein level or mRNA level in the first biological sample is measured or estimated and compared to a standard t-PALP protein level or mRNA level, the standard being taken from a second biological sample obtained from an individual not having the disorder or being determined by averaging levels from a population of individuals not having a disorder of the circulatory system. As will be appreciated in the art, once a standard t-PALP protein level or mRNA level is known, it can be used repeatedly as a standard for comparison.

By “biological sample” is intended any biological sample obtained from an individual, body fluid, cell line, tissue culture, or other source which contains t-PALP protein or mRNA. As indicated, biological samples include body fluids (such as sera, plasma, urine, synovial fluid and spinal fluid) which contain free t-PALP protein, circulatory system tissue, and other tissue sources found to express complete or mature t-PALP or a t-PALP receptor. Methods for obtaining tissue biopsies and body fluids from mammals are well known in the art. Where the biological sample is to include mRNA, a tissue biopsy is the preferred source.

The present invention is useful for diagnosis or treatment of various circulatory system-related disorders in mammals, preferably humans. Such disorders include any disregulation of circulatory cell function including, but not limited to, diseases related to thrombosis, which is characterized by hypercoagulation of blood cells. t-PALP may be employed to prevent proximal extension of deep-venous thrombosis or the recurrence of pulmonary embolisms, which are characterized by lodging of a blood clot in a pulmonary artery with subsequent obstruction of blood supply to the lung parenchyma. t-PALP may also be employed to help prevent the recurrence of cerebral or other systemic embolisms. The enzyme of the present invention may also be used to treat high risk patients, such as those who have congestive heart failure, acute myocardial infarction or cardiomyopathy to prevent the development of deep-vein thrombosis or pulmonary embolism. t-PALP may also be employed as a long-term therapy for the occasional patient who has recurrent thrombosis or embolism while on the drug Warfarin.

Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding the t-PALP protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase cha in reaction (RT-L CR).

Assaying t-PALP protein levels in a biological sample can occur using antibody-based techniques. For example, t-PALP protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)). Other antibody-based methods useful for detecting t-PALP protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

In addition to assaying t-PALP protein levels in a biological sample obtained from an individual, t-PALP protein can also be detected in vivo by imaging. Antibody labels or markers for in vivo imaging of t-PALP protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A t-PALP protein-specific antibody or antibody fragment which has been labeled with an appropriate detectable imaging moiety, such as a radioisotope (for example, ¹³¹I, ¹¹²In, ^{99m}Tc), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain t-PALP protein. In vivo tumor imaging is described in S. W. Burchiel et al., “Immunopharmacokinetics of Radiolabeled Antibodies and Their Fragments” (Chapter 13 in Tumor Imaging: The Radiochernical Detection of Cancer, S. W. Burchiel and B. A. Rhodes, eds., Masson Publishing Inc. (1982)).

Treatment

As noted above, t-PALP polynucleotides and polypeptides are useful for diagnosis of conditions involving abnormally high or low expression of t-PALP activities. Given the cells and tissues where t-PALP is expressed as well as the activities modulated by t-PALP, it is readily apparent that a substantially altered (increased or decreased) level of expression of t-PALP in an individual compared to the standard or “normal” level produces pathological conditions related to the bodily system(s) in which t-PALP is expressed and/or is active.

It will also be appreciated by one of ordinary skill that, since the t-PALP protein of the invention is related to t-PA the mature secreted form of the protein may be released in soluble form from the cells which express the t-PALP by proteolytic cleavage. Therefore, when t-PALP mature form is added from an exogenous source to cells, tissues or the body of an individual, the protein will exert its physiological activities on its target cells of that individual.

Therefore, it will be appreciated that conditions caused by a decrease in the standard or normal level of t-PALP activity in an individual, particularly disorders of the circulatory system, can be treated by administration of t-PALP polypeptide (in the form of the mature, secreted protein). Thus, the invention also provides a method of treatment of an individual in need of an increased level of t-PALP activity comprising administering to such an individual a pharmaceutical composition comprising an amount of an isolated t-PALP polypeptide of the invention, particularly a mature form of the t-PALP protein of the invention, effective to increase the t-PALP activity level in such an individual.

t-PALP may also be employed in combinations. compositions, and methods for treating thrombic disease. For example, the enzyme of the present invention may be combined with a thrombolytic agent to work in a complementary fashion to dissolve blood clots, resulting in decreased reperfusion times and increased reocclusion times in patients. The thrombolytic agent dissolves the clot while t-PALP prevents thrombin from regenerating the clot. This combination allows the administration of a thrombolytic agent at a considerably lower dosage than if given alone, therefore, allowing the prevention of undesirable side-effects associated with the use of a high level of thrombolytic agent, for example, bleeding complications.

Formulations

The t-PALP polypeptide composition will be formulated and dosed in a fashion consistent with good medical practice, taking into account the clinical condition of the individual patient (especially the side effects of treatment with t-PALP polypeptide alone), the site of delivery of the t-PALP polypeptide composition, the method of administration, the scheduling of administration, and other factors known to practitioners. The “effective amount” of t-PALP polypeptide for purposes herein is thus determined by such considerations.

As a general proposition, the total pharmaceutically effective amount of t-PALP polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the t-PALP polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the t-PALP of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The t-PALP polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release t-PALP polypeptide compositions also include liposomally entrapped t-PALP polypeptide. Liposomes containing t-PALP polypeptide are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal t-PALP polypeptide therapy.

For parenteral administration, in one embodiment, the t-PALP polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the t-PALP polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The t-PALP polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of t-PALP polypeptide salts.

t-PALP polypeptide to be used for therapeutic administration must be sterile. Sterility is readily accomplished by filtration through sterile filtration membranes (e.g., 0.2 micron membranes). Therapeutic t-PALP polypeptide compositions generally are placed into a container having a sterile access port, for example, an intravenous solution bag or vial having a stopper pierceable by a hypodermic injection needle.

t-PALP polypeptide ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous t-PALP polypeptide solution, and the resulting mixture is lyophilized. The infusion solution is prepared by reconstituting the lyophilized t-PALP polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

Agonists and Antagonists—Assays and Molecules

The invention also provides a method of screening compounds to identify those which enhance or block the action of t-PALP on cells, such as its interaction with t-PALP-binding molecules. An agonist is a compound which increases the natural biological functions of t-PALP or which functions in a manner similar to t-PALP, while antagonists decrease or eliminate such functions.

In another aspect of this embodiment the invention provides a method for identifying a protein which binds specifically to a t-PALP polypeptide. For example, the t-PALP polypeptide may be bound to a solid support so that binding molecules solubilized from cells are bound to the column and then eluted and characterized according to routine methods.

In the assay of the invention for agonists or antagonists, a cellular compartment, such as a membrane or a preparation thereof, may be prepared from a cell that expresses a molecule that binds t-PALP. The preparation is incubated with labeled t-PALP in the absence or the presence of a candidate molecule which may be a t-PALP agonist or antagonist. The ability of the candidate molecule to bind the binding molecule is reflected in decreased binding of the labeled ligand. Molecules which bind gratuitously, i.e., without inducing the effects of t-PALP on binding the t-PALP binding molecule, are most likely to be good antagonists. Molecules that bind well and elicit effects that are the same as or closely related to t-PALP are agonists.

t-PALP-like effects of potential agonists and antagonists may by measured, for instance, by determining activity of a second messenger system following interaction of the candidate molecule with a cell or appropriate cell preparation, and comparing the effect with that of t-PALP or molecules that elicit the same effects as t-PALP. Second messenger systems that may be useful in this regard include but are not limited to AMP guanylate cyclase, ion channel or phosphoinositide hydrolysis second messenger systems.

Another example of an assay for t-PALP antagonists is a competitive assay that combines t-PALP and a potential antagonist with recombinant t-PALP receptor molecules under appropriate conditions for a competitive inhibition assay. t-PALP can be labeled, such as by radioactivity, such that the number of t-PALP molecules bound to a receptor molecule can be determined accurately to assess the effectiveness of the potential antagonist.

Potential antagonists include small organic molecules, peptides, polypeptides and antibodies that bind to a polypeptide of the invention and thereby inhibit or extinguish its activity. Potential antagonists also may be small organic molecules, a peptide, a polypeptide such as a closely related protein or antibody that binds the same sites on a binding molecule, such as a receptor molecule, without inducing t-PALP-induced activities, thereby preventing the action of t-PALP by excluding t-PALP from binding.

Other potential antagonists include antisense molecules. Antisense technology can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression.” CRC Press, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of t-PALP. The antisense RNA oligonucleotide hybridizes to the mRNA in vivo and blocks translation of the mRNA molecule into t-PALP polypeptide. The oligonucleotides described above can also be delivered to cells such that the antisense RNA or DNA may be expressed in vivo to inhibit production of t-PALP protein.

The agonists and antagonists may be employed in a composition with a pharmaceutically acceptable carrier, e.g., as described above.

The antagonists may be employed for instance to inhibit t-PALP activities such as fibrin binding. By inhibition of fibrin binding, a t-PALP antagonist may decrease the efficacy of t-PALP enzymatic activity. Such an inhibition may of interest if it is desirable to negatively alter t-PALP activity in an indirect manner. Rather than directly targeting the active site of the t-PALP enzyme, it may be of interest to alter the activity of the enzyme by targeting its fibrin-binding activity. Furthermore, t-PALP may be of use in regulating the proteolytic activity plasminogen. An antagonist which functions by directly binding to the t-PALP active site may reduce the local concentration of functional plasminogen in a given system. Such a capability may desired as an effective means of ameliorating a current treatment procedure which has artificially increased the effective concentration of plasminogen. In addition, the use of such a t-PALP antagonist may be used effectively to treat a system which has a congenitally increased level of t-PALP, and in turn, plasminogen activity. Similarly, antibodies against t-PALP may be employed to bind to and inhibit t-PALP activity to treat the same or a related condition. Any of the above antagonists may be employed in a composition with a pharmaceutically acceptable carrier, e.g., as hereinafter described.

Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a t-PALP protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

### Example 1

### Expression and Purification of “His-tagged” t-PALP in E. coli

The bacterial expression vector pQE9 (pD10) is used for bacterial expression in this example. (QIAGEN, Inc., 9259 Eton Avenue, Chatsworth, Calif., 91311). pQE9 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that an inserted DNA fragment encoding a polypeptide expresses that polypeptide with the six His residues (i.e., a “6× His tag”) covalently linked to the amino terminus of that polypeptide.

The DNA sequence encoding the desired portion of the t-PALP protein comprising the mature form of the t-PALP amino acid sequence is amplified from the deposited cDNA clone using PCR oligonucleotide primers which anneal to the amino terminal sequences of the desired portion of the t-PALP protein and to sequences in the deposited construct 3′ to the cDNA coding sequence. Additional nucleotides containing restriction sites to facilitate cloning in the pQE9 vector are added to the 5′ and 3′ primer sequences, respectively.

For cloning the mature form of the t-PALP protein, the 5′ primer has the sequence 5′ GGCCGACATGTCTGGAGGCTGTTTCTGG 3′ (SEQ ID NO:11) containing the underlined Afl III restriction site followed by 17 nucleotides of the amino terminal coding sequence of the mature t-PALP sequence in SEQ ID NO:2. One of ordinary skill in the art would appreciate, of course, that the point in the protein coding sequence where the 5′ primer begins may be varied to amplify a DNA segment encoding any desired portion of the complete t-PALP protein shorter or longer than the mature form of the protein. The 3′ primer has the sequence 5′ GGCGGAAGCTTATTAGGCCCCAGGAGTCCCGGC 3′ (SEQ ID NO:12) containing the underlined Hind III restriction site followed by 22 nucleotides complementary to the 3′ end of the coding sequence of the t-PALP DNA sequence in FIG. 1.

The amplified t-PALP DNA fragment and the vector pQE9 are digested with Afl III and Hind III and the digested DNAs are then ligated together. Insertion of the t-PALP DNA into the restricted pQE9 vector places the t-PALP protein coding region downstream from the IPTG-inducible promoter and in-frame with an initiating AUG and the six histidine codons.

The ligation mixture is transformed into competent E. coli cells using standard procedures such as those described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing t-PALP protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-β-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH 8. The cell debris is removed by centrifugation, and the supernatant containing the t-PALP is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QIAexpressionist, 1995, QIAGEN, Inc., supra). Briefly the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the t-PALP is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins can be eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

The following alternative method may be used to purify t-PALP expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells ware then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000× g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000× g centrifugation for 15 min., the pellet is discarded and the t-PALP polypeptide-containing supernatant is incubated at 4° C. overnight to allow further GuHCl extraction.

Following high speed centrifugation (30,000× g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded t-PALP polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the t-PALP polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the t-PALP polypeptide (determined, for instance, by 16% SDS-PAGE) are then pooled.

The resultant t-PALP polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

### Example 2

### Cloning and Expression of t-PALP Protein in a Baculovirus Expression System

In this illustrative example, the plasmid shuttle vector pA2 is used to insert the cloned DNA encoding complete protein, including its naturally associated secretory signal (leader) sequence, into a baculovirus to express the mature t-PALP protein, using standard methods as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites such as Bam HI, Xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

The cDNA sequence encoding the full length t-PALP protein in the deposited clone, including the AUG initiation codon and the naturally associated leader sequence shown in SEQ ID NO:2, is amplified using PCR oligonucleotide primers corresponding to the 5′ and 3′ sequences of the gene. The 5′ primer has the sequence 5′

5′GGCCGGGATCCGCCATCATGCTGTTGGCCTGGGTAC 3′(SEQ ID NO:13)

containing the underlined Bam HI restriction enzyme site, an efficient signal for initiation of translation in eukaryotic cells, as described by Kozak, M., J. Mol. Biol. 196:947-950 (1987), followed by 25 of nucleotides of the sequence of the complete t-PALP protein shown in FIG. 1, beginning with the AUG initiation codon. The 3′ primer has the sequence 5′ GGCCGGGTACCTT

ATTAGGCCCCAGGAGTCCCGGC 3′ (SEQ ID NO:14) containing the underlined Asp 718 restriction site followed by 24 nucleotides complementary to the 3′ noncoding sequence in FIG. 1.

The amplified fragment is isolated from a 1% agarose gel using a commercially available kit (“Geneclean,” BIO 101 Inc., La Jolla, Calif.). The fragment then is digested with Bam HI and Asp 718 and again is purified on a 1% agarose gel. This fragment is designated herein F1.

The plasmid is digested with the restriction enzymes Bam HI and Asp 718 and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB 101 or other suitable E. coli hosts such as XL-1 Blue (Statagene Cloning Systems, La Jolla, Calif.) cells are transformed with the ligation mixture and spread on culture plates. Bacteria are identified that contain the plasmid with the human t-PALP gene by digesting DNA from individual colonies using Bam HI and Asp 718 and then analyzing the digestion product by gel electrophoresis. The sequence of the cloned fragment is confirmed by DNA sequencing. This plasmid is designated herein pA2t-PALP.

Five μg of the plasmid pA2t-PALP is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84: 7413-7417 (1987). One μg of BaculoGold™ virus DNA and 5 μg of the plasmid pA2t-PALP are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 mil of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-t-PALP.

To verify the expression of the t-PALP gene Sf9 cells are grown in Grace's medium supplemented with 10% heat-inactivated FBS. The cells are infected with the recombinant baculovirus V-t-PALP at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the mature form of the t-PALP protein and thus the cleavage point and length of the naturally associated secretory signal peptide.

### Example 3

### Cloning and Expression of t-PALP in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QC1-3 cells. mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (GS) (Murphy et al., Biochem. J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985)). Multiple cloning sites, e.g., with the restriction enzyme cleavage sites Bam HI, Xba I and Asp 718, facilitate the cloning of the gene of interest. The vectors contain in addition the 3′ intron, the polyadenylation and termination signal of the rat preproinsulin gene.

The expression plasmid, pt-PALPHA, is made by cloning a portion of the cDNA encoding the mature form of the t-PALP protein into the expression vector pcDNAI/Amp or pcDNAIII (which can be obtained from Invitrogen, Inc.). The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al., Cell 37: 767 (1984). The fusion of the HA tag to the target protein allows easy detection and recovery of the recombinant protein with an antibody that recognizes the HA epitope. pcDNAIII contains, in addition, the selectable neomycin marker.

### Example 3(a)

### Cloning and Expression in COS Cells

A DNA fragment encoding the complete t-PALP polypeptide is cloned into the polylinker region of the vector so that recombinant protein expression is directed by the CMV promoter. The plasmid construction strategy is as follows. The t-PALP cDNA of the deposited clone is amplified using primers that contain convenient restriction sites, much as described above for construction of vectors for expression of t-PALP in E. coli. Suitable primers include the following, which are used in this example. The 5′ primer, containing the underlined Bam HI site, a Kozak sequence, an AUG start codon, and 25 nucleotides of the 5′ coding region of the complete t-PALP polypeptide, has the following sequence: 5′ GGCCGGG

ATCCGCCATCATGCTGTTGGCCTGGGTAC 3′ (SEQ ID NO:15). The 3′ primer, containing the underlined Asp 718 and 24 of nucleotides complementary to the 3′ coding sequence immediately before the stop codon, has the following sequence: 5′

5′GGCCGGGTACCTTATTAGGCCCCAGGAGTCCCGGC 3′(SEQ ID NO:16).

The PCR amplified DNA fragment and the vector, pcDNAI/Amp, are digested with Bam HI and Asp 718 and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the complete t-PALP polypeptide

For expression of recombinant t-PALP, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989). Cells are incubated under conditions for expression of t-PALP by the vector.

Expression of the t-PALP-HA fusion protein is detected by radiolabeling and immunoprecipitation, using methods described in, for example Harlow et al., Antibodies: A Laboratory Manual, 2nd Ed.; Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of t-PALP polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rouse Sarcoma Virus (Cullen, et al., Molecular and Cellular Biology, March 1985:438-447) plus a fragment isolated from the enhancer of the immediate early gene of human cytomegalovirus (CMV) (Boshart et al., Cell 41:521-530 (1985)). Downstream of the promoter are the following single restriction enzyme cleavage sites that allow the integration of the genes: BamHI, Xba I, and Asp718. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the t-PALP polypeptide in a regulated way in mammalian cells (Gossen, M., & Bujard, H. 1992, Proc. Natl. Acad. Sci. USA 89:5547-5551). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes Bam HI and Asp 718 and then dephosphorylated using calf intestinal phosphates by procedures known in the art. The vector is then isolated from a 1% agarose gel.

The DNA sequence encoding the t-PALP polypeptide is amplified using PCR oligonucleotide primers corresponding to the 5′ and 3′ sequences of the desired portion of the gene. The 5′ primer containing the underlined Barn HI site, a Kozak sequence, an AUG start codon, and 25 nucleotides of the 5′ coding region of the t-PALP polypeptide, has the following sequence: 5′ GGCCGGGATCCGC

CATCATGCTGTTGGCCTGGGTAC 3′ (SEQ ID NO:15). The 3′ primer, containing the underlined Asp 718 and 24 of nucleotides complementary to the 3′ coding sequence immediately before the stop codon as shown in FIG. 1 (SEQ ID NO:1), has the following sequence: 5′ GGCCGGGTACCTTATTAGGCCCCA

GGAGTCCCGGC 3′ (SEQ ID NO:16).

The amplified fragment is digested with the endonucleases Bam HI and Asp 718 and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 4

### Tissue Distribution of t-PALP mRNA Expression

Northern blot analysis was carried out to examine t-PALP gene expression in human tissues using methods described by, among others, Sambrook et al., cited above. A cDNA probe containing the entire nucleotide sequence of the t-PALP protein (SEQ ID NO:1) was labeled with ³²P using the rediprime™ DNA labeling system (Amersham Life Science), according to manufacturer's instructions. After labeling, the probe was purified using a TE Select-D G50 spin column (5 prime-3 prime, Inc.) according to manufacturer's recommendations. The purified labeled probe was then used to examine various human tissues for t-PALP mRNA.

Multiple Tissue Northern (MTN) blots containing various human tissues (H) or human immune system tissues (IM) were obtained from Clontech and were examined with the labeled probe using ExpressHyb™ hybridization solution (Clontech) according to manufacturer's protocol number PT 1190-1. Following hybridization and washing, the blots were mounted and exposed to film at −70° C. overnight, and films developed according to standard procedures.

The Northern blot experiments described above indicated expression of 2.5 kb t-PALP message in the following tissues: heart, brain, placenta, lung, liver, skeletal muscle, kidney, pancreas, spleen, thymus, prostate, testis, ovary, small intestine, colon, and peripheral blood leukocytes.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a member selected from the group consisting of:
(a) a nucleotide sequence encoding the t-PALP polypeptide having the amino acid sequence at positions −21 to 242 of SEQ ID NO:2, or the complete amino acid sequence encoded by the cDNA clone contained in ATCC Deposit No. 209023;
(b) a nucleotide sequence encoding the t-PALP polypeptide having the amino acid sequence at positions −20 to 242 of SEQ ID NO:2, or the complete amino acid sequence excepting the N-terminal methionine encoded by the cDNA clone contained in ATCC Deposit No. 209023;
(c) a nucleotide sequence encoding the mature t-PALP polypeptide having the amino acid sequence at positions 1 to 242 of SEQ ID NO:2, or as encoded by the cDNA clone contained in ATCC Deposit No. 209023;
(d) a nucleotide sequence encoding the kringle domain of the t-PALP polypeptide having the amino acid sequence at positions 4 to 63 of SEQ ID NO:2, or as encoded by the cDNA clone contained in ATCC Deposit No. 209023;
(e) a nucleotide sequence encoding the protease domain of the t-PALP polypeptide having the amino acid sequence at positions 64 to 242 of SEQ ID NO:2, or as encoded by the cDNA clone contained in ATCC Deposit No. 209023;
(f) a nucleotide sequence complementary to any of the nucleotide sequences in (a), (b), (c), (d) or (e) above.

2. The nucleic acid molecule of claim 1 wherein said polynucleotide has the complete nucleotide sequence in FIG. 1 (SEQ ID NO:1).

3. The nucleic acid molecule of claim 1 wherein said polynucleotide has the nucleotide sequence in FIG. 1 (SEQ ID NO:1) encoding the t-PALP polypeptide having the amino acid sequence of positions −20 to 242 of SEQ ID NO:2.

4. The nucleic acid molecule of claim 1 wherein said polynucleotide has the nucleotide sequence in FIG. 1 (SEQ ID NO:1) encoding the mature t-PALP polypeptide having the amino acid sequence at positions 1 to 242 in SEQ ID NO:2.

5. An isolated nucleic acid molecule comprising a polynucleotide having a nucleotide sequence at least 95% identical to a member selected from the group consisting of:
(a) a nucleotide sequence encoding a polypeptide comprising the amino acid sequence of residues n to 242 of SEQ ID NO:2, where n is an integer in the range of −20 to 64;
(b) a nucleotide sequence encoding a polypeptide comprising the amino acid sequence of residues −20 to m of SEQ ID NO:2, where m is an integer in the range of 230 to 241;
(c) a nucleotide sequence encoding a polypeptide having the amino acid sequence consisting of residues n to m of SEQ ID NO:2, where n and m are integers as defined respectively in (a) and (b) above; and
(d) a nucleotide sequence encoding a polypeptide consisting of a portion of the complete t-PALP amino acid sequence encoded by the cDNA clone contained in ATCC Deposit No. 209023 wherein said portion excludes from 1 to about 63 amino acids from the amino terminus;
(e) a nucleotide sequence encoding a polypeptide consisting of a portion of the complete t-PALP amino acid sequence encoded by the cDNA clone contained in ATCC Deposit No. 209023 wherein said portion excludes from 1 to about 11 amino acids from the carboxy terminus; and
(f) a nucleotide sequence encoding a polypeptide consisting of a portion of the complete t-PALP amino acid sequence encoded by the cDNA clone contained in ATCC Deposit No. 209023 wherein said portion includes a combination of any of the amino terminal and carboxy terminal deletions in (d) and (e), above.

6. The nucleic acid molecule of claim 1 wherein said polynucleotide has the complete nucleotide sequence of the cDNA clone contained in ATCC Deposit No. 209023.

7. The nucleic acid molecule of claim 1 wherein said polynucleotide has the nucleotide sequence encoding the t-PALP polypeptide having the complete amino acid sequence excepting the N-terminal methionine encoded by the cDNA clone contained in ATCC Deposit No. 209023.

8. The nucleic acid molecule of claim 1 wherein said polynucleotide has the nucleotide sequence encoding the mature t-PALP polypeptide having the amino acid sequence encoded by the cDNA clone contained in ATCC Deposit No. 209023.

9. An isolated nucleic acid molecule comprising a polynucleotide which hybridizes under stringent hybridization conditions to a polynucleotide having a nucleotide sequence identical to a nucleotide sequence in (a), (b), (c), (d) or (e) of claim 1 wherein said polynucleotide which hybridizes does not hybridize under stringent hybridization conditions to a polynucleotide having a nucleotide sequence consisting of only A residues or of only T residues.

10. An isolated nucleic acid molecule comprising a polynucleotide which encodes the amino acid sequence of an epitope-bearing portion of a t-PALP polypeptide having an amino acid sequence in (a), (b), (c) or (d) of claim 1.

11. The isolated nucleic acid molecule of claim 10, which encodes an epitope-bearing portion of a t-PALP polypeptide wherein the amino acid sequence of said portion is selected from the group of sequences in SEQ ID NO:2 consisting of: about Ser-1 to about His-10 in SEQ ID NO:2; about Glu-14 to about Leu-23 in SEQ ID NO:2; about Arg-50 to about Trp-60 in SEQ ID NO:2; about Pro-70 to about Gln-86 in SEQ ID NO:2; about Ala-98 to about Val-107 in SEQ ID NO:2; about Leu-117 to about Gln-126 in SEQ ID NO:2; about Arg-134 to about Gly-146 in SEQ ID NO:2; about Ser-172 to about Gln-182 in SEQ ID NO:2; about Gln-185 to about Arg-194 in SEQ ID NO:2; about Thr-206 to about Val-216 in SEQ ID NO:2; and about Thr-222 to about Thr-231 in SEQ ID NO:2.

12. A method of making a recombinant vector comprising inserting an isolated nucleic acid molecule of claim 1 into a vector.

13. A recombinant vector produced by the method of claim 12.

14. A method of making a recombinant host cell comprising operably linking the nucleic acid molecule of claim 1 to a foreign expression control element.

15. A recombinant host cell produced by the method of claim 14.

16. A recombinant method for producing a t-PALP polypeptide, comprising culturing the recombinant host cell of claim 15 under conditions such that said polypeptide is expressed and recovering said polypeptide.

17. An isolated t-PALP polypeptide comprising an amino acid sequence at least 95% identical to a member selected from the group consisting of:
(a) the amino acid sequence at positions −20 to 242 of SEQ ID NO:2, or the complete t-PALP amino acid sequence excepting the N-terminal methionine encoded by the cDNA clone contained in ATCC Deposit No. 209023;
(b) the amino acid sequence of the mature form of the t-PALP polypeptide having the amino acid sequence at positions 1 to 242 of SEQ ID NO:2, or as encoded by the cDNA clone contained in the ATCC Deposit No. 209023;
(c) the amino acid sequence of the kringle domain of the t-PALP polypeptide having the amino acid sequence at positions 4 to 63 of SEQ ID NO:2, or as encoded by the cDNA clone contained in the ATCC Deposit No. 209023; and
(d) the amino acid sequence of the mature t-PALP polypeptide having the amino acid sequence at positions 64 to 242 of SEQ ID NO:2, or as encoded by the cDNA clone contained in the ATCC Deposit No. 209023; and
(e) the amino acid sequence of an epitope-bearing portion t-PALP.

18. The isolated polypeptide of claim 17 comprising an epitope-bearing portion of the t-PALP protein, wherein said portion is selected from the group consisting of: a polypeptide comprising amino acid residues from about Ser-1 to about His-10 in SEQ ID NO:2; about Glu-14 to about Leu-23 in SEQ ID NO:2; about Arg-50 to about Trp-60 in SEQ ID NO:2; about Pro-70 to about Gln-86 in SEQ ID NO:2; about Ala-98 to about Val-107 in SEQ ID NO:2; about Leu-117 to about Gln-126 in SEQ ID NO:2; about Arg-134 to about Gly-146 in SEQ ID NO:2; about Ser-172 to about Gln-182 in SEQ ID NO:2; about Gln-185 to about Arg-194 in SEQ ID NO:2; about Thr-206 to about Val-216 in SEQ ID NO:2; and about Thr-222 to about Thr-231 in SEQ ID NO:2.

19. An isolated antibody that binds specifically to a t-PALP polypeptide of claim 17.

20. An isolated nucleic acid molecule comprising a polynucleotide having a sequence at least 95% identical to a sequence selected from the group consisting of:
(a) the nucleotide sequence of a portion of the sequence shown in FIG. 1 (SEQ ID NO:1) wherein said portion comprises at least 50 contiguous nucleotides from 1 to 110 or from 630 to 750;
(b) the nucleotide sequence of a portion of the sequence shown in FIG. 1 (SEQ ID NO:1) wherein said portion consists of nucleotides 1 to 2000, 1 to 1500, 1 to 1000, 1 to 500, 1 to 250, 250 to 2000, 250 to 1500, 250 to 1000, 250 to 500, 500 to 2000, 500 to 1500, 500 to 1000. 1000 to 2000, or 1000 to 1500; and
(c) a nucleotide sequence complementary to any of the nucleotide sequences in (a) or (b) above.


SEQUENCE LISTING (16 sequences)

SEQ ID NO: 1 - DNA (genomic), 2329 bp; single-stranded, linear
  CDS 124..913
  sig_peptide 124..184
  mat_peptide 187..913
ttaccagaacagcataacaagggcaggtctgactgcaagctgggactgggaggcagagccgccgccaagggggcctcggttaaacactggtcgttcaatcacctgcaagacgaagaggcaaggatgctgttggcctgggtacaagcattcctcgtcagcaacatgctcctagcagaagcctatggatctggaggctgtttctgggacaacggccacctgtaccgggaggaccagacctcccccgcgccgggcctccgctgcctcaactggctggacgcgcagagcgggctggcctcggcccccgtgtcgggggccggcaatcacagttactgccgaaacccggacgaggacccgcgcgggccctggtgctacgtcagtggcgaggccggcgtccctgagaaacggccttgcgaggacctgcgctgtccagagaccacctcccaggccctgccagccttcacgacagaaatccaggaagcgtctgaagggccaggtgcagatgaggtgcaggtgttcgctcctgccaacgccctgcccgctcggagtgaggcggcagctgtgcagccagtgattgggatcagccagcgggtgcggatgaactccaaggagaaaaaggacctgggaactctgggctacgtgctgggcattaccatgatggtgatcatcattgccatcggagctggcatcatcttgggctactcctacaagagggggaaggatttgaaagaacagcatgatcagaaagtatgtgagagggagatgcagcgaatcactctgcccttgtctgccttcaccaaccccacctgtgagattgtggatgagaagactgtcgtggtccacaccagccagactccagttgaccctcaggagggcagcaccccccttatgggccaggccgggactcctggggcctgagcccccccagtgggcaggagcccatgcagacactggtgcaggacagcccaccctcctacagctaggaggaactaccactttgtgttctggttaaaaccctaccactcccccgcttttttggcgaatcctagtaagagtgacagaagcaggtggccctgtgggctgagggtaaggctgggtagggtcctaacagtgctccttgtccatcccttggagcagattttgtctgtggatggagacagtggcagctcccacagtgatgctgctgctaagggcttccaaacattgcctgcacccctggaactgaaccagggatagacggggagctcccccaggctcctctgtgctttactaagatggctcagtctccactgtgggcttgagtggcatacactgttattcatggttaaggtaaagcaggtcaagggatggcattgaaaaaatatatttagtttttaaaatatttgggatggaactccctactgacctctgacaactggaaacgagtttgtactgaagtcagaactttgggttgggaatgagatctaggttgtggctgctggtatgcttcagcttgctggcaatgatgtgccttgacaaccgtgggccaggcctgggcccagggactcttcctgtttcataaggaaaggaagaattgcactgagcattccacttaggaagaggatagagaaggatctgctccgcctttggccacaggagcagaggcagacctgggatgccccagtttctcttcagggatggatagtgacctgtcttcattttgcacaggtaagagagtagttagctaacctatgggaattatactgtggggccttgtgagctgcttctaagaggctaacctggaaactaagctcagaggcaaggtaataaagcacttcagggcttgctccccaagtgggcctgatttagcaggtggtctgcgggcgtccaggtcagcaccttcctgtagggcactggggctagggtcacagcccctaactcataaagcaatcaaagaaccattagaaagggctcattaagccttttggacacaggaccccagagaggaaaaagtgacttgcccaaggtcgtaagcaagctactggcatggcaagagcccagcttcctgacggagcgcaacatttctccactgcactgtgctagcagctcagcagggcctctaacctgtgatgtcacactcaagaggccttggcagctcctagccatagagcttcctttccagaacccttccactgcccaatgtggagacaggggttagtggggctttctatggagccatctgctttggggacctagacctcaggtggtctcttggtgttagtgatgctggagaagagaatattactggtttctacttttctataaaggcatttctctataaaaaaaaaaaaaa

SEQ ID NO: 2 - protein, 263 aa
MLLAWVQAFLVSNMLLAEAYGSGGCFWDNGHLYREDQTSPAPGLRCLNWLDAQSGLASAPVSGAGNHSYCRNPDEDPRGPWCYVSGEAGVPEKRPCEDLRCPETTSQALPAFTTEIQEASEGPGADEVQVFAPANALPARSEAAAVQPVIGISQRVRMNSKEKKDLGTLGYVLGITMMVIIIAIGAGIILGYSYKRGKDLKEQHDQKVCEREMQRITLPLSAFTNPTCEIVDEKTVVVHTSQTPVDPQEGSTPLMGQAGTPGA

SEQ ID NO: 3 - protein, 372 aa
YVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 4 - DNA (genomic), 250 bp; single-stranded, linear
attgcactgagcattccacttaggaagaggatagagaaggatctgctccgcctttggccacaggagcagaggcagacctgggatgccccatttctcttcagggatggatagtgacctgtcttcattttgcacaggtaagagagtagttagctaacctatgggaattatactgtggggccttgtagctgcttctaagaggctaacctggaaactaagctcagaggcaaggtaataaagcacttcagggctt

SEQ ID NO: 5 - DNA (genomic), 247 bp; single-stranded, linear
atagagaaatgcctttatagaaaagtagaaaccagtaatattctcttctccagcatcactaacaccaagagaccacctgaggtctaggtccccaaagcagatggctccatagaaagccccactaacccgtctccacattgggcagtggaagggttctggaaaggaagctctatggctaggagctgccaaggcctcttgagtgtgacatcacaggttagaggccctgctgagctgctagcacagtgca

SEQ ID NO: 6 - DNA (genomic), 461 bp; single-stranded, linear
aattcggcaagagtaacagcataacaagggtaggtctgactgcagctgggactgggaggcagagcacgccaagggggcctcggttaaacactggtcgttcaatcacctgcaaacgaggaggcaaggatgctgttggcctgggtacagcattcctggtcagcaacatgctcctagcgtaagcctatggatctggaggctgtttctgggacaacggccactgtacccggaggaccagaccttcccggccggtcctcgtgcctcaactggctggacgcgcagggctgcctgggcccccttttcggtcaaatttcacagtttacttcgaaaccgggacggggccgtgggggccctggtggttagtttggggtcgggttttcttaaaaaaggtttttggggccggttttcggaaccatttcggttgaattttttagggaaatttcaggagttttttaagggccatt

SEQ ID NO: 7 - DNA (genomic), 399 bp; single-stranded, linear
ggcaagttgcagaactggaaacgagtttgtacagaagtcagaactttgggttaggaatgagatctaggttgtggctgctggtatgcttcattgctggcaataatgtgccttgacaaccgtgggccaggcctgggaccagggactcttcctgtttcataaggaaaggaagaattgcactgagcattccacttaggaagaggatagagcaaggaatctgctccgctttggccacaggagcagaggcagacctgggatgccccagttctctttcagggatgggatagtgacctgtcttacattttgcacaggtaaagagagttagttagctaacctattgggctttattacttggggcttgtgagctgctttttaagaggttaacctggaactaaagttcag

SEQ ID NO: 8 - DNA (genomic), 334 bp; single-stranded, linear
taattcggcaagggacaggtctgactgcagctgggactgggaggcagagccgtcaagggggcctcggttaaacactggtcgttcaatcacctgcaacgagaggcaaggatgctgttggcctgggtacaagcattcctgtcagcaacatgctcctagcagaaagcctatggatctgggaggctgtttctgggacaacggccacctgtaccggaggaccagacctccccggccgggccttccgtggccttcaattggtttgacgtggcaaaggggcttgtctggcccttttgggggaaaattacaagttttaattgtcccggaaaacctggagagg

SEQ ID NO: 9 - DNA (genomic), 472 bp; single-stranded, linear
aattcggcagagggagagggagatgcagcgaatcactctgcccttgtctgccttcaccaaccccacctgtgagattgtggatgagaagactgtcgtggtccacaccagccagactccagttgaccctcaggagggcagcaccccccttatgggaccaggccggggactcctggggcctgagccccccagtggggcaggagccatggcagacactggtgcaggacagccaccctccttacagctagggggaactaccactttgtgtttctggtttaaaaccctaccactcccggattttttggcggattccttagttaagagtacagaagcaggtgggcctatggcttggagggtaaggtggggtagggttcctaaaagtgggttcttggttgctcctgggaggaagattttggttttggtggggacagtggcagtttccacaggttgttgtgttaaggggttcaaaaaattg

SEQ ID NO: 10 - DNA (genomic), 291 bp; single-stranded, linear
gggcacgagatgaactccaaggagaaaaaggacctgggaactctgggtatgacggtcccccacccctgcccttgttgggattcatcaagagatgtcatttgctgattgtctagggtgtggctaatgggaccttgtgtcctatccttggcaggctacgtgctgggcattaccatgatggtgatcatcattgccatcggagctggcatcatcttgggctactctacaagaggtcagtagcttctcttctgggccctcttaggaggaggggaggaaggtacacaaagtcaaact

SEQ ID NO: 11 - DNA (genomic), 28 bp; single-stranded, linear
ggccgacatgtctggaggctgtttctgg

SEQ ID NO: 12 - DNA (genomic), 33 bp; single-stranded, linear
ggcggaagcttattaggccccaggagtcccggc

SEQ ID NO: 13 - DNA (genomic), 36 bp; single-stranded, linear
ggccgggatccgccatcatgctgttggcctgggtac

SEQ ID NO: 14 - DNA (genomic), 35 bp; single-stranded, linear
ggccgggtaccttattaggccccaggagtcccggc

SEQ ID NO: 15 - DNA (genomic), 36 bp; single-stranded, linear; identical to SEQ ID NO: 13

SEQ ID NO: 16 - DNA (genomic), 35 bp; single-stranded, linear; identical to SEQ ID NO: 14